(19) United States
(12) Patent Application Publication
(10) Pub. No.: US 2025/0161463 A1
(43) Pub. Date: May 22, 2025

(54) 2-DEOXY-GLUCOSE (2DG)- OR TREHALOSE-TERMINATED DENDRIMER COMPOSITION

(71) Applicant: Washington State University, Pullman, WA (US)

(72) Inventors: Anjali Sharma, Pullman, WA (US); Rishi Sharma, Pullman, WA (US); Anubhav Dhull, Pullman, WA (US); Anu Rani, Pullman, WA (US)

(73) Assignee: Washington State University, Pullman, WA (US)

(21) Appl. No.: 18/955,682

(22) Filed: Nov. 21, 2024

Related U.S. Application Data
(60) Provisional application No. 63/602,334, filed on Nov. 22, 2023.
(60) Provisional application No. 63/569,661, filed on Mar. 25, 2024.

(51) Int. Cl. A61K 47/54; A61K 47/60

(52) U.S. Cl. CPC A61K 47/549; A61K 47/60

(57) ABSTRACT

A dendrimer that includes: (a) a central core; and (b)(i) at least one 2-deoxyglucose-surfaced branching units conjugated to the central core; or (b)(ii) at least one trehalose-surfaced branching units conjugated to the central core; or (b)(iii) at least one 2-deoxyglucose-surfaced branching units conjugated to the central core and at least one trehalose-surfaced branching units conjugated to the central core.

### CROSS REFERENCE TO RELATED APPLICATION

This application claims the benefit of U.S. Provisional Appl. No. 63/602,334, filed Nov. 22, 2023, and U.S. Provisional Appl. No. 63/569,661, filed Mar. 25, 2024, both of which are incorporated by reference herein.

### BACKGROUND

Recent advances in drug design and synthetic chemistry have led to the discovery of thousands of drug composition alternatives that can be used for treatment of deleterious maladies such as, but not limited to, brain diseases, cancer and inflammation induced processes. However, a large majority of such new molecules (compositions) do not move beyond initial clinical phases to become active therapies. A problematic hurdle for potential useful therapies is a lack of effective formulation strategies that aid a composition's solubility, stability, enhance its transport across epithelial and endothelial barriers encountered within the body, and allow selective targeted drug accumulation and retention in diseased tissue(s).

Accordingly, there is a need for more sophisticated therapeutic drug conjugates that can actively target and selectively accumulate in diseased and/or symptomatic tissue and produce a controlled drug release.

### SUMMARY

The embodiments herein are directed to new dendrimer compositions which are formulated to aid in methodologies for targeting specific drug delivery to tissues of interest. In particular, disclosed herein are 2-deoxyglucose (2DG)-surfaced dendrimers and trehalose-surfaced dendrimers.

In one embodiment, the disclosed beneficial composition includes a 2-deoxyglucose (2DG) dendrimer nanoplatform wherein the peripheral layer of the dendrimer comprises 2-deoxyglucose. More specifically, wherein a mixed layer glycodendrimer has an outer layer fully configured of 2-deoxyglucose. The 2-deoxyglucose (2DG) dendrimer nanoplatform allows for ease of scalability and flexibility, in vivo stability, low immunogenicity, and high aqueous solubility for single or multiple drugs targeted delivery.

Accordingly, such a new dendrimer having 2DG on the periphery, has favorable therapeutic effects against different diseases and can be administered in a variety of modalities. Moreover, the dendrimer composition disclosed herein has beneficial uses as nanocarrier for a) drug delivery, b) solubility enhancement by conjugation and complexation, c) diagnosis, d) targeting, and e) imaging purposes.

It is to be appreciated that an important and beneficial aspect of the disclosed mixed layer 2DG-dendrimer herein is that the installed sugar heads (2DG) on the periphery have therapeutic value. The 2DG, which is an analogue of glucose but lacks a hydroxy group at position 2, has demonstrated cancer and neuroinflammation targeting, cancer therapeutic, and anti-inflammatory properties.

In another embodiment, there is disclosed a mixed layer dendrimer molecule comprising a plurality of layers of building blocks in an interior and a surface (i.e, peripheral) layer of one or more trehalose (tre) units.

### BRIEF DESCRIPTION OF THE DRAWINGS

FIG. 1: Example structure of a generation3 (G3) 2-deoxyglucose dendrimer nanoplatform (2DG-dendrimer), as disclosed herein, wherein the outer layer (G3) is comprised of 2DG, inner layer (G2) is comprised of a 3,4,5-trihydroxybenzoic acid-based building block, and the innermost layer (G1) is 8-arm acetylene-terminated-polyamidoamine generation 1 dendrimer serving as a core.

FIG. 2: shows the complete structure of 2DG-dendrimer.

FIG. 3: shows an example structure of a G3 trehalose dendrimer nanoplatform (tre-dendrimer), as disclosed herein, wherein the outer layer (G3) is comprised of trehalose, inner layer (G2) comprised of a 3,4,5-trihydroxybenzoic acid-based building block, and the innermost layer (G1) is 8-arm acetylene-terminated-polyamidoamine generation 1 dendrimer serving as a core.

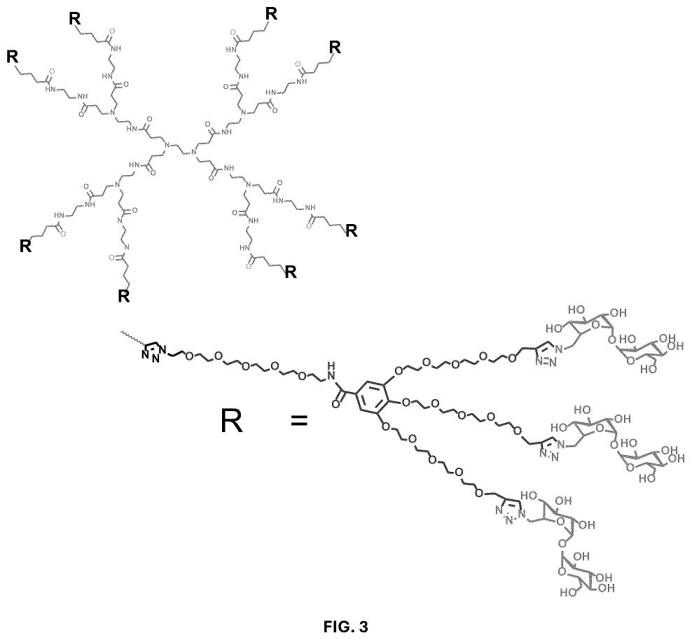

FIG. 4: shows the complete structure of tre-dendrimer.

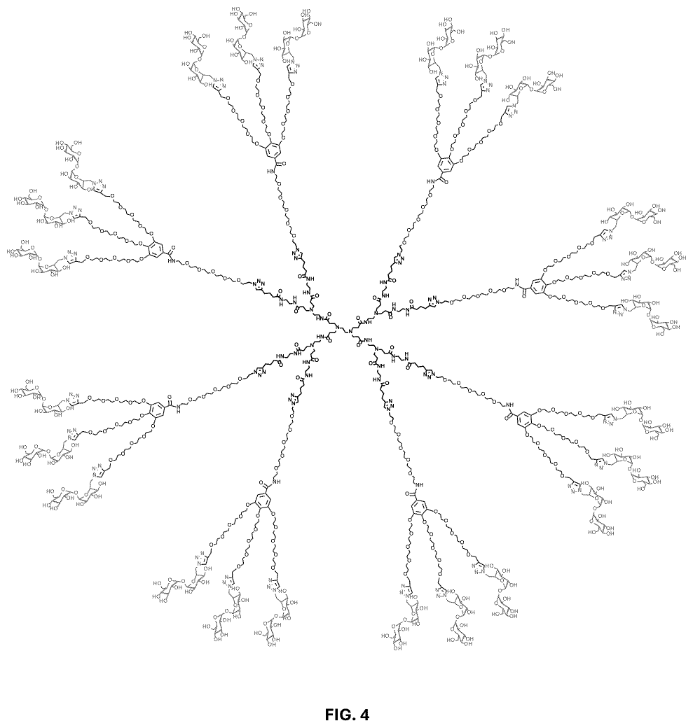

FIG. 5: Example of a 2-deoxyglucose (2DG)-surfaced dendrimer around a cyclodextrin core.

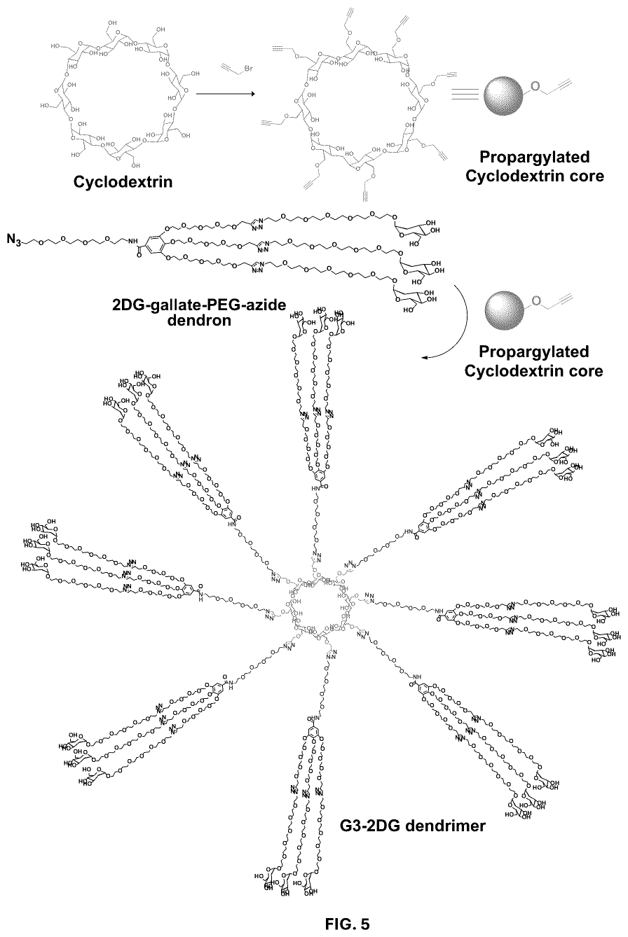

FIG. 6: Examples of Generation (G) 3 2DG-surfaced dendrimers with cyclodextrin core and different dendrons.

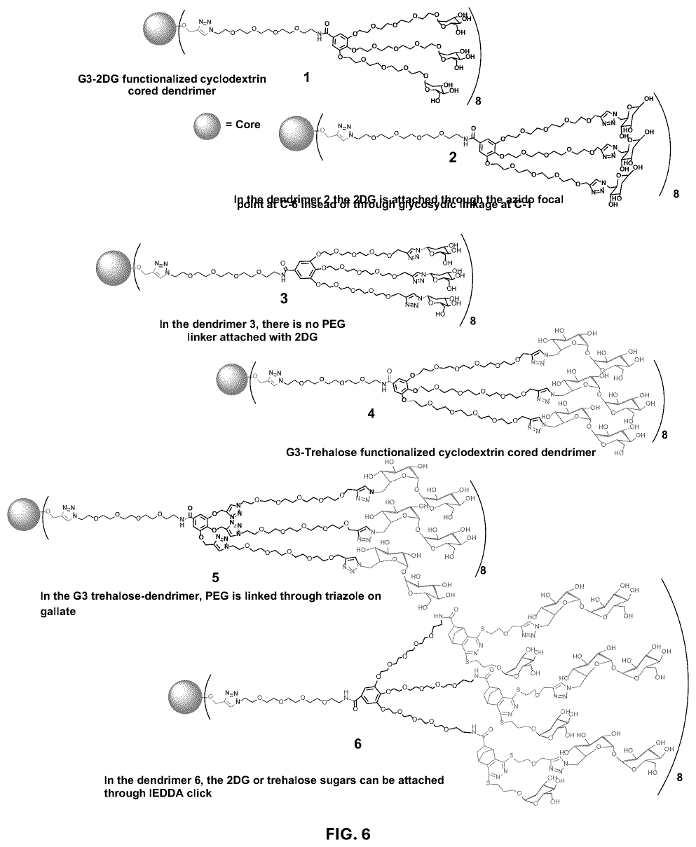

FIG. 7: Example of a G4 2DG-dendrimer with cyclodextrin core.

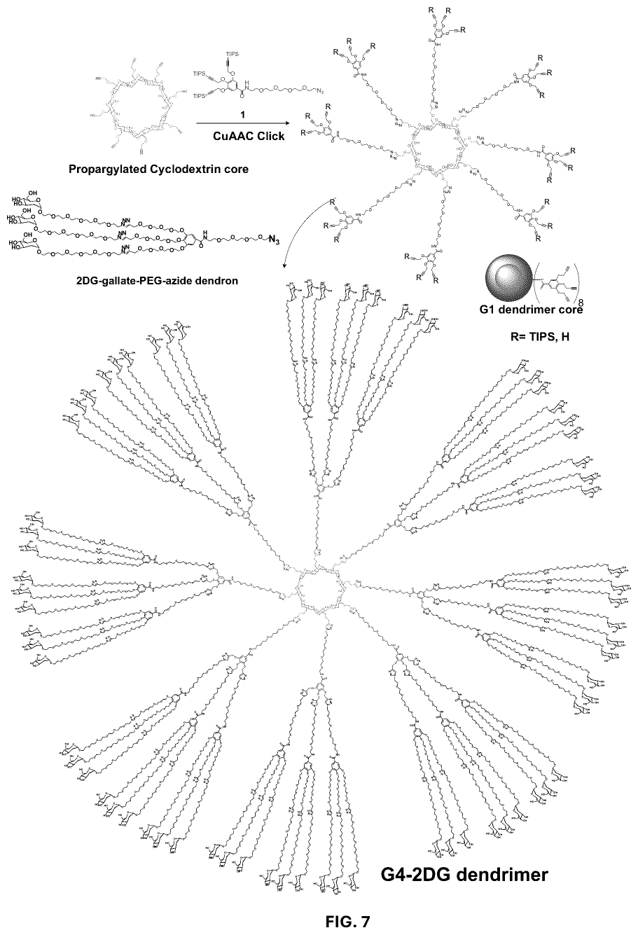

FIG. 8: Examples of G4 dendrimers surfaced with 2DG or trehalose sugars with various cores.

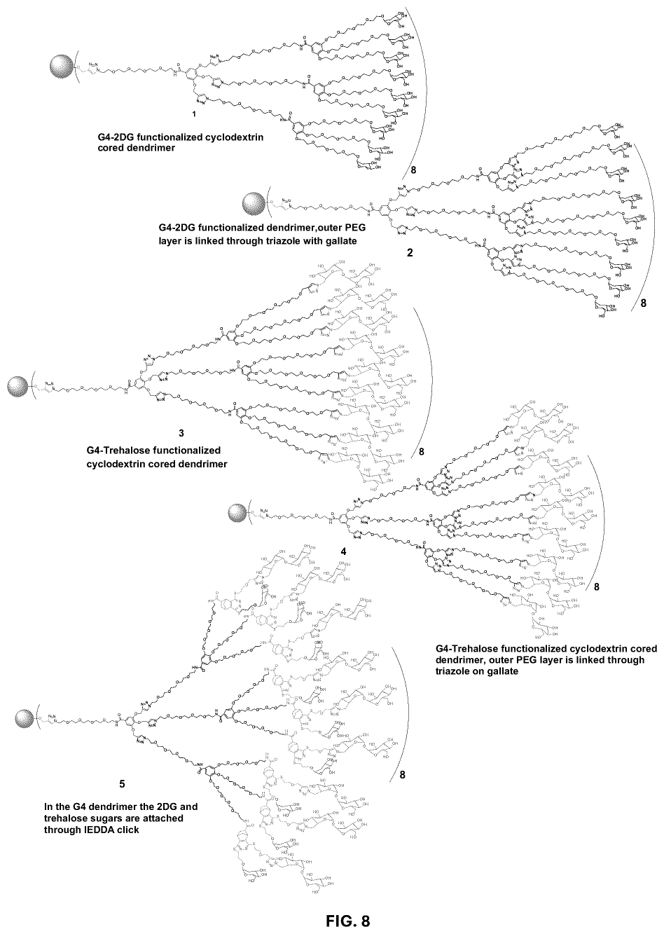

FIG. 9: Examples of trehalose or 2DG-surfaced dendrons (arms of dendrimers).

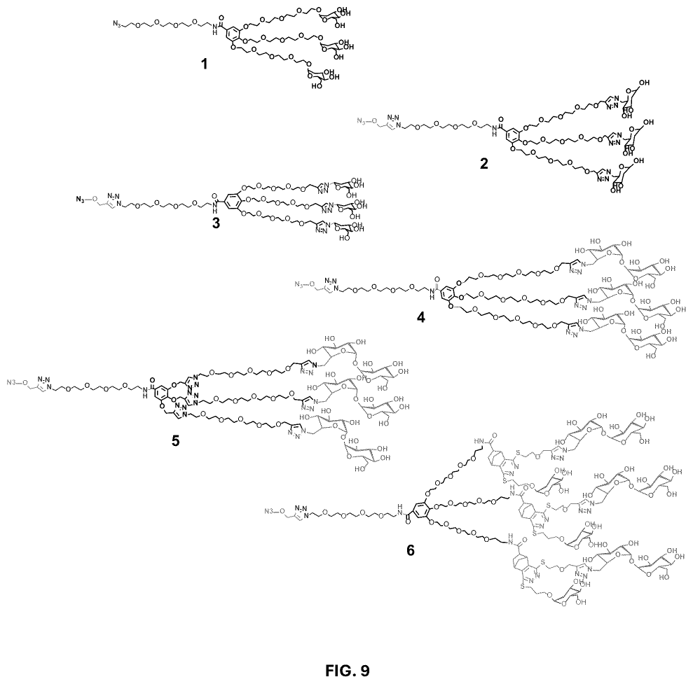

FIG. 10: More examples of trehalose or 2DG-surfaced dendrons (arms of dendrimers).

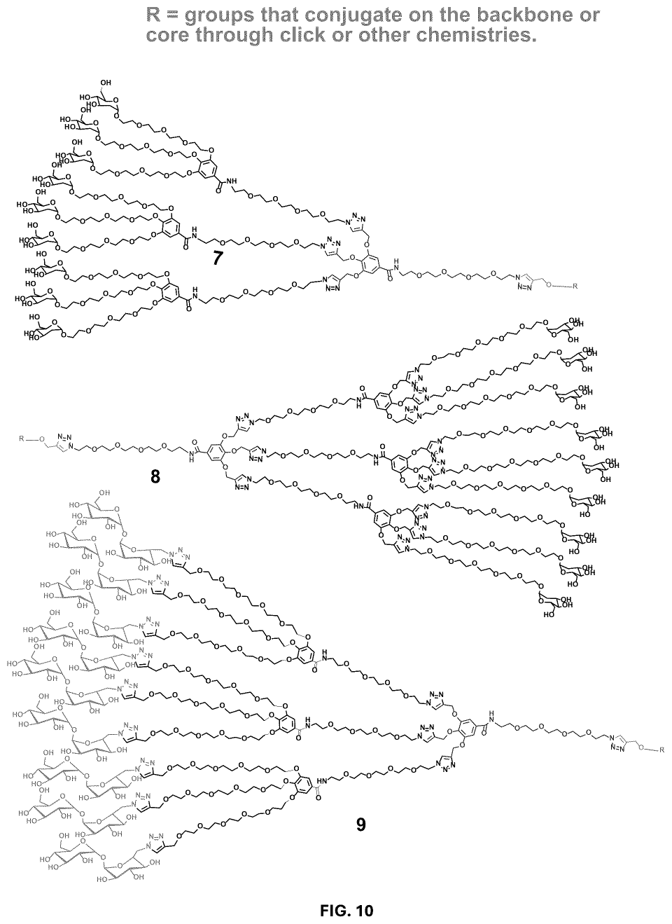

FIG. 11: More examples of trehalose or 2DG-surfaced dendrons (arms of dendrimers).

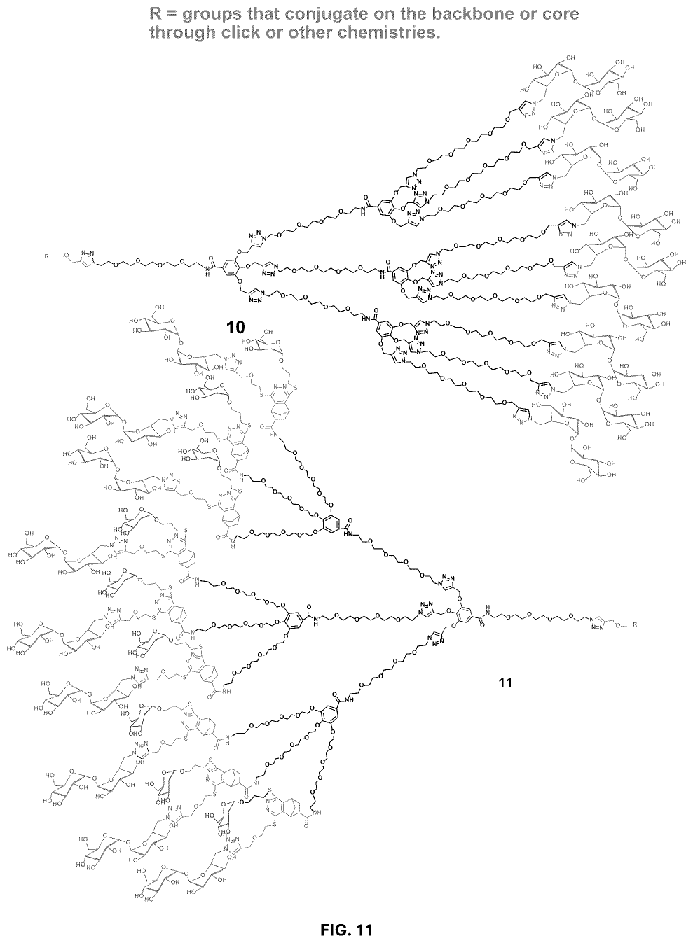

FIG. 12: Examples of cores/backbones to which 2DG or trehalose can be attached either directly or through linkers or in the form of dendrons presented in FIGS. 6 and 7.

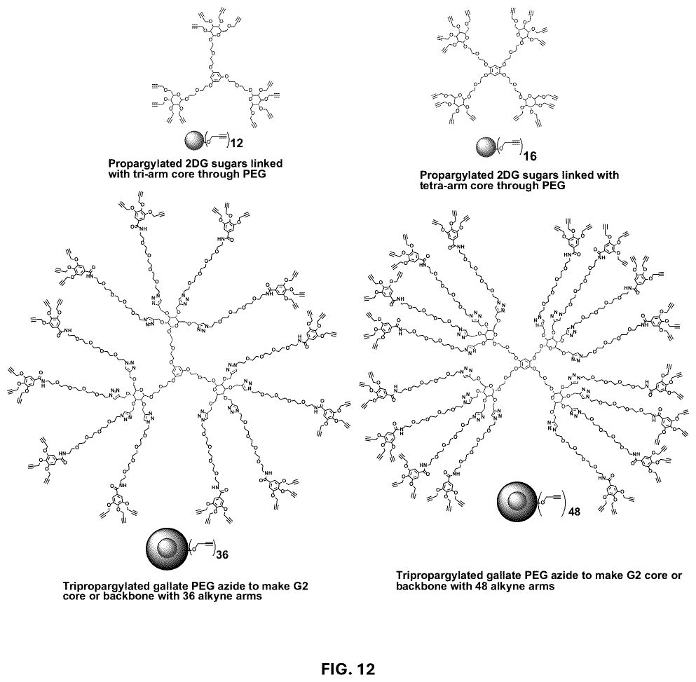

FIG. 13: Example of a 2DG-surfaced dendrimer with 12 arm core.

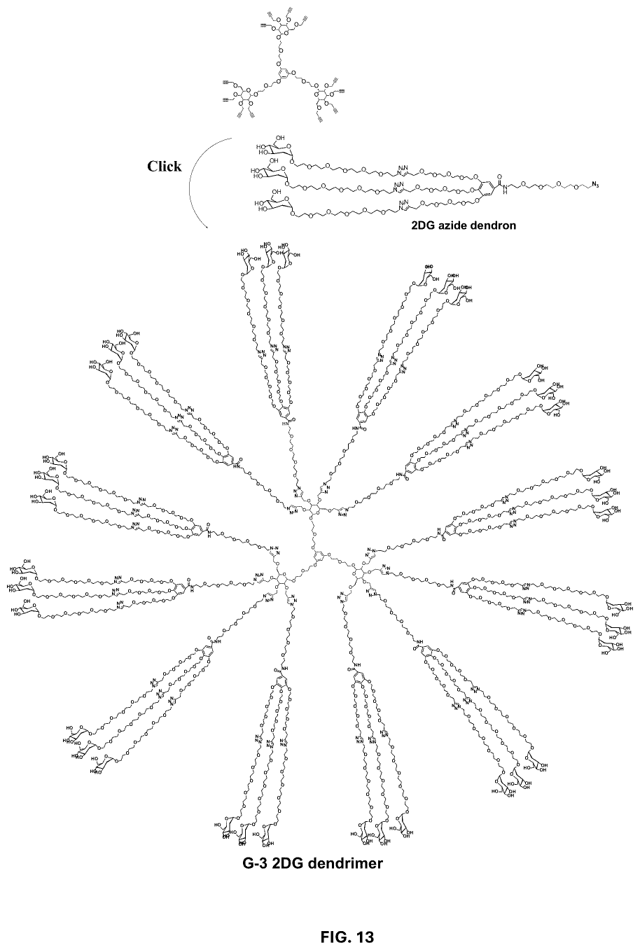

FIG. 14: Example of a 2DG-surfaced dendrimer with 16 arm core.

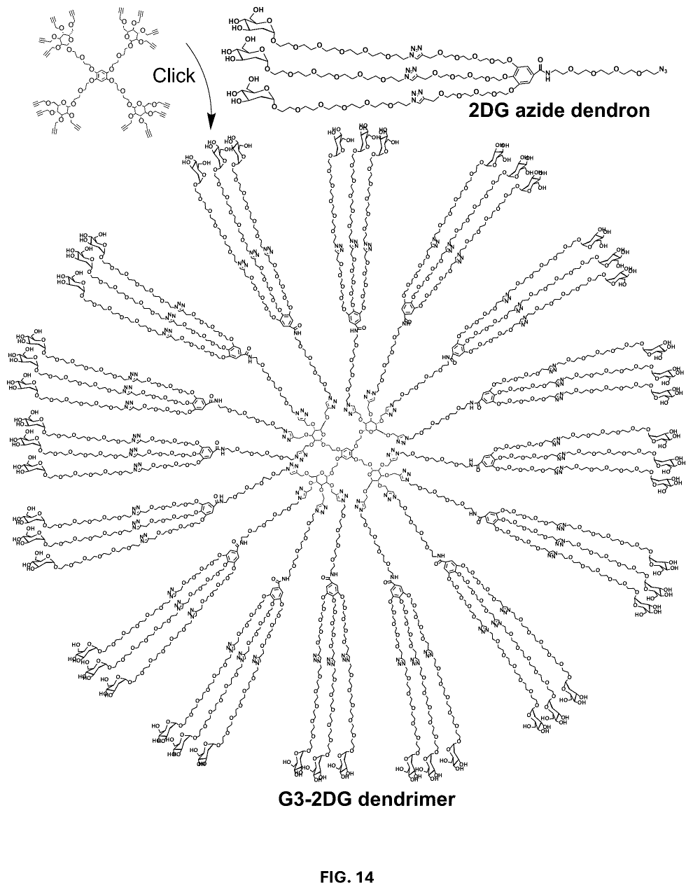

FIG. 15: Example of a 2DG-surfaced dendrimer with 36 arm core.

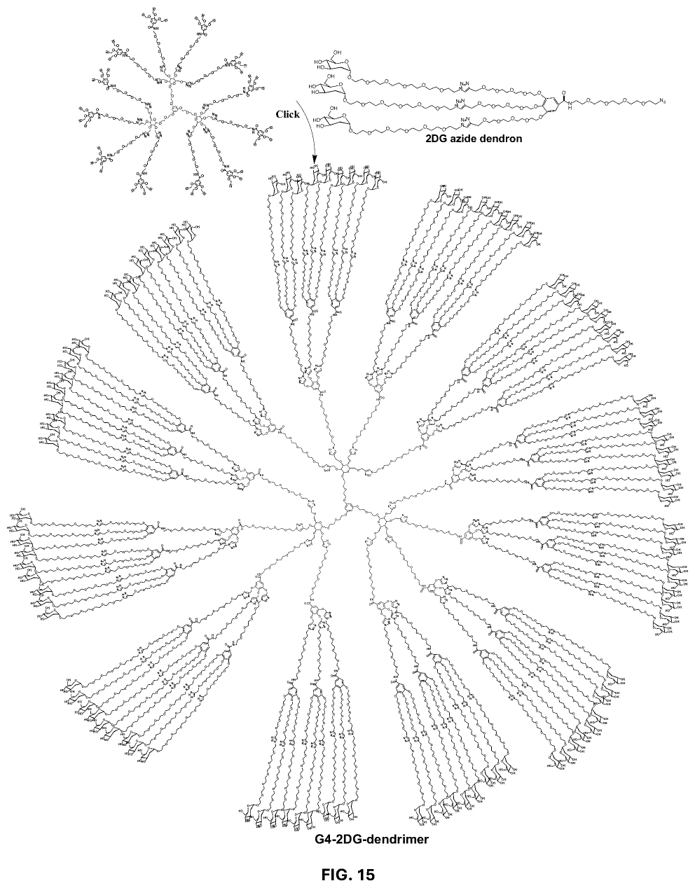

FIG. 16: Example of a 2DG-surfaced dendrimer synthesized using strain promoted click chemistry.

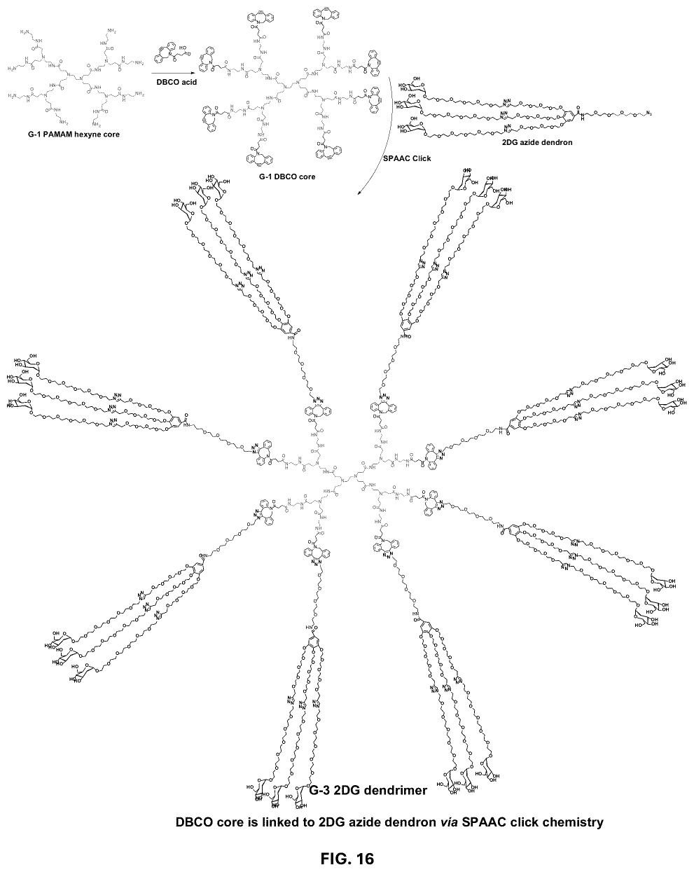

FIG. 17: Example of a trehalose-surfaced dendrimer synthesized using strain promoted click chemistry.

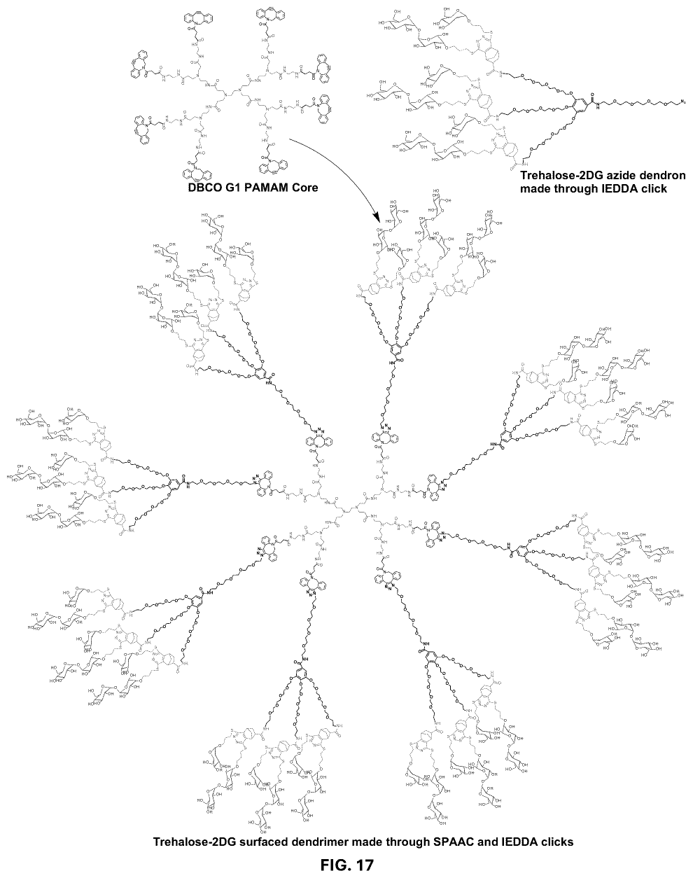

FIG. 18: Examples of various cores and backbones used to build 2DG- and trehalose-surfaced dendrimers.

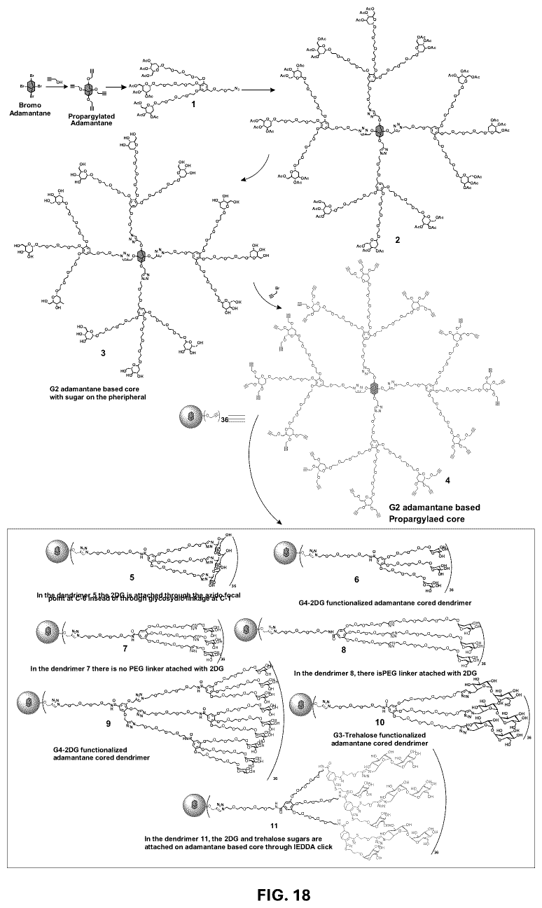

FIG. 19: Example of a Janus-type dendrimer with 2DG and/or trehalose surface groups or a combination with other sugars.

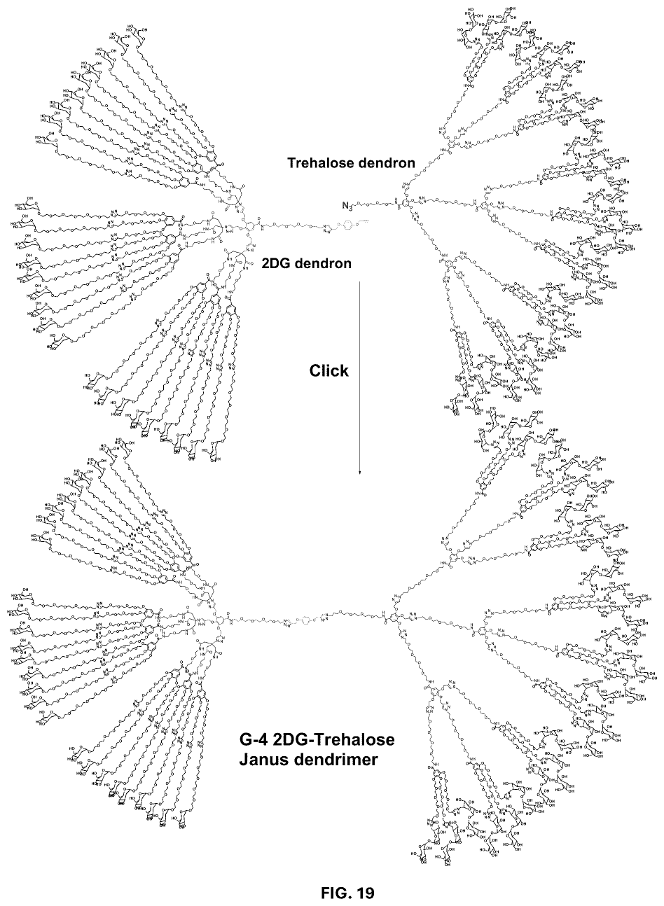

FIG. 20: Example of a Janus-type dendrimer with 2DG and/or trehalose surface groups or a combination with other sugars.

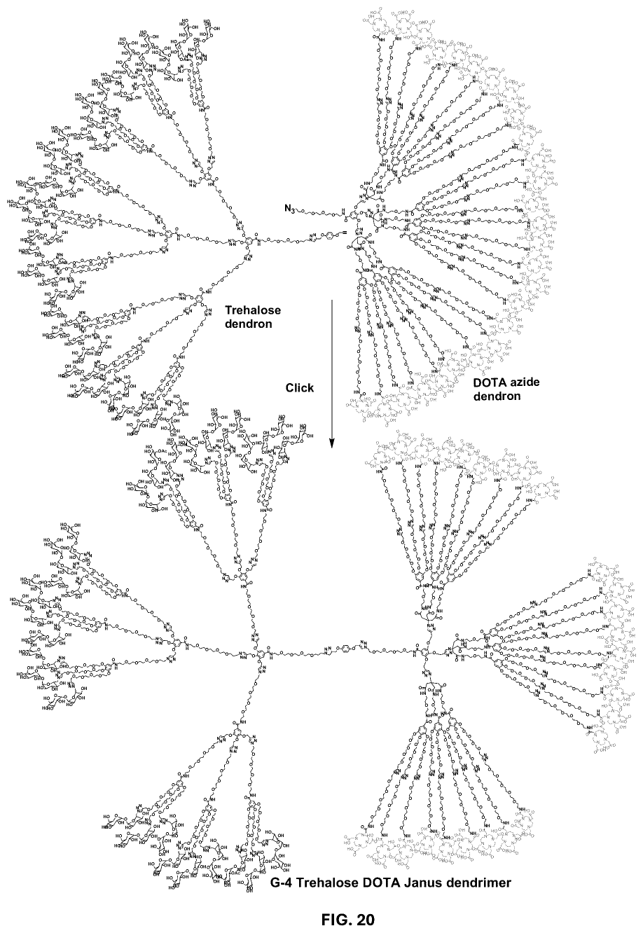

FIG. 21. Synthetic route to 2 Deoxy Glucose-Dendrimer (2DG-D). A) Synthetic route to a clickable dendron containing three 2DG molecules; B) Synthesis of 2DG-D from 8-arm alkyne core; C) structure of 2DG-D depicting 24-2DG molecules at the surface. Reagents and conditions: (i) K₂CO₃, DMF, 16 h, 50° C., MW, 60%; (ii) LiOH·H₂O, THF: H₂O, RT, 48 h, 95%; (iii) BF₃·OEt, Dry DCM, 24 h, RT, 50%; (iv) CuSO₄·H₂O, Sod. Ascorbate, DMF, 40° C., MW, 10 h, 90%; (v) EDC.HCl, HOBt, DCM, RT, 1.5h, 90%; (vi) NaOMe, MeOH, RT, 24 h, 89% (vii) EDC.HCl, DMAP, DMF, 16 h, RT, 84%; (viii) CuSO₄·5H₂O, Sodium ascorbate, 40° C., MW, 10 h, 90%; (ix) NaOMe, MeOH, RT, 24 h, 90%; (x) CuSO₄·5H₂O, Sodium ascorbate, 80° C., MW, 15 h, 95%.

FIG. 22. Characterization of 2DG-dendrimer. A) ¹H NMR spectra of intermediate dendron, hyper-core, protected and deprotected 2DG-D representing the appearance and disappearance of characteristic protons; B) HPLC chromatogram of 2DG-D showing >99% purity; C) Table representing the physicochemical properties of 2DG-D.

FIG. 23. Reproducible synthesis of 2DG-D and Cy5 conjugation. A) The figure shows the reproducibility in the synthesis of multiple 5 g-scale batches of 2DG-D via ¹H NMR and HPLC. The ¹H NMR spectra of 3 batches (5 g scale) show characteristic protons from 2DG-D. The HPLC chromatographs show reproducibility in the purity of these batches with all these batches showing >99% purity.

FIG. 24. Schematic representation for the divergent synthesis of 2DG-D. The scheme represents the step wise synthesis of 2DG-D using an inside-outwards divergent synthetic approach.

FIG. 25. ¹H NMR Characterization of 2DG-dendrimer. ¹H NMR spectra showing the appearance and disappearance of characteristic proton signals at each step of the divergent synthesis of 2DG-D.

FIG. 26. HPLC Characterization and Plasma Stability of 2DG-D. A) HPLC chromatogram showing a shift in retention time during 2DG-D synthesis in a divergent fashion. B) HPLC chromatogram showing stability of 2DG-D through similar retention times when incubated in plasma at physiological temperature for 7 days.

FIG. 27: Synthesis of Trehalose dendrimer. Reagent and conditions (i) DMF, NaH, RT, 3 h, 85% (ii) LiOH, THF-H₂O, RT, 24 h, 78% (iii) CuSO₄·H₂O, Sodium ascorbate, DMF, 50° C., 12 h, 90% (iv) EDC.HCl, HOBT, DCM, RT 85% (v) NaOMe, MeOH, RT, 16 h, 92% (vi) CuSO₄·5H₂O, Sodium ascorbate, 80° C., MW, 15 h, 85%. (vii) NaOMe, MeOH, RT, 16 h, 90% (vi) CuSO₄·5H₂O, Sodium ascorbate, 80° C., MW, 15 h, 86%.

FIG. 28 (A) ¹H NMR spectra of intermediate dendrons, hyper-core, protected and deprotected Tre-D dendrimers representing the appearance and disappearance of characteristic protons; (B) HPLC chromatogram of Tre-D showing >99% purity; (C) ¹H NMR data and HPLC chromatogram demonstrate reproducibility is the structure and purity among three different batches.

### DETAILED DESCRIPTION

In the description herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

### Dendrimers—General

The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation.

Methods for making dendrimers are known to those of skill in the art and generally involve a two-step iterative reaction sequence that produces concentric shells (generations) of dendritic units around a central initiator core (e.g., ethylenediamine-cores). Each subsequent growth step represents a new “generation” of polymer.

Dendrimers are usually synthesized according to methods allowing controlling their structure at every stage of construction. The dendritic structures are mostly synthesized by two main different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups.

In other embodiments, dendrimers are prepared using convergent methods, in which dendrimers are built from small molecules that end up at the surface of the sphere, and reactions proceed inward building inward and are eventually attached to a core.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or active agents via click chemistry, employing one or more of Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or active agents via amidation, esterification, etherification, or similar chemistries.

In some embodiments, dendrimer synthesis replies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

### 2-deoxyglucose-Surfaced Dendrimers and Trehalose-Surfaced Dendrimers

In one embodiment, there is disclosed herein a dendrimer that includes:
(a) a central core; and
(b)(i) at least one 2-deoxyglucose-surfaced branching units conjugated to the central core; or
(b)(ii) at least one trehalose-surfaced branching units conjugated to the central core; or
(b)(iii) at least one 2-deoxyglucose-surfaced branching units conjugated to the central core and at least one trehalose-surfaced branching units conjugated to the central core.

In certain embodiments, all the branching units present in the dendrimer are 2-deoxyglucose-surfaced branching units or trehalose-surfaced branching units.

In certain embodiments, the central core comprises polyamidoamine, cyclodextrin, polylysine, 2,2-bismethylolpropionic, tetrazine, poly(propylene imine), polyethylene glycol, glycol, or adamantane.

In certain embodiments, the branching units include at least one focal point selected from triazole, gallic acid, or an amino acid.

In certain embodiments, the branching units include repeating units selected from polyethylene glycol, an alkane, a peptide, or an amino acid.

In certain embodiments, the dendrimer includes 1 to 200 2-deoxyglucose-surfaced branching units, or 1 to 200 trehalose-surfaced branching units.

In another embodiment, there is disclosed herein a dendrimer that includes:
(a) a core;
(b) at least one interior layer comprising repeating units; and
(c)(i) 2-deoxyglucose terminal surface groups attached to an outermost interior layer; or
(c)(ii) trehalose terminal surface groups attached to an outermost interior layer; or
(c)(iii) 2-deoxyglucose terminal surface groups attached to an outermost interior layer and trehalose terminal surface groups attached to an outermost interior layer.

In certain embodiments, the entire periphery of the dendrimer consists of 2-deoxyglucose terminal surface groups or trehalose terminal groups.

In certain embodiments, the core comprises polyamidoamine, cyclodextrin, polylysine, 2,2-bismethylolpropionic, tetrazine, poly(propylene imine), polyethylene glycol, glycol, or adamantane, or molecules with multiple hydroxyl, carboxylic acid, alkynes, azides, amines, strained alkynes, tetrazine functional groups.

In certain embodiments, the at least one interior layer includes at least one focal point selected from triazole, gallic acid, an amino acid, a peptide, or a linear polymer.

In certain embodiments, the repeating units are selected from polyethylene glycol, an amino acid, a peptide, or a linear polymer.

In certain embodiments, the dendrimer includes 1 to 15 interior layers, more particularly 1 to 10 interior layers.

In a further embodiment, there is disclosed a dendrimer that includes one to ten generations, wherein at least one of generation 1 to generation 10 includes 2-deoxyglucose groups or trehalose groups, wherein the —OH of the 2-deoxyglucose groups or the —HO of the trehalose groups are not attached to any other moiety or atom. In other words, free (i.e., exposed) 2DG groups can be attached at any location on the dendrimer including the core, an interior layer or at the dendrimer molecule surface.

In an additional embodiment, there is disclosed herein a dendrimer that includes a plurality of generations, wherein generation 1 includes a polyamidoamine bearing hexyne arms, generation 2 includes a gallic acid building blocks-based layer; and at least one additional generation includes 2-deoxyglucose surface groups or trehalose surface groups. In certain embodiments, generation 3, generation 4, or generation 5 includes the 2-deoxyglucose surface groups or trehalose surface groups.

In another embodiment, there is disclosed herein a composition that includes a 2-deoxyglucose-terminated dendrimer configured around a 2-armed core to 200-armed core.

In a further embodiment, there is disclosed herein a composition that includes a trehalose-terminated dendrimer configured around a 2-armed core to 200-armed core.

In certain embodiments, there is disclosed herein a dendrimer having a surface (i.e., periphery) that has only 2DG groups or trehalose groups.

In certain embodiments, the dendrimer can include a plurality of linkers on the core, each linker having from 1 to 50 hydrocarbon units.

In certain embodiments, the dendrimers can include a plurality of linkages in the dendrimer molecule backbone selected from disulfide, ester, ether, carbonate, carbamate, thiol, thioester, cathepsin sensitive, maleimidomethyl, thioether, hydrazine, glucuronide bond, hydrazides, N-alkyl, ethyl, hydroxymethyl, and amide.

In certain embodiments, the dendrimer can include a 2,2-bismethylolpropionic (bis-MPA) or polylysine core, PAMAM dendron as the second layer, gallic acid as the third layer, PEG as the fourth layer, and 2DG and/or trehalose at the surface.

Also disclose herein are compositions that include the 2DG- or trehalose-surfaced dendrimers crosslinked with at least one other agent such as hydrogels, linear polymers, hyperbranched polymers, glycopolymers, biopolymers, or implants. Illustrative glycopolymers include hyaluronic acid, chitosan, and dextran.

Also disclose herein are compositions that include the 2DG- or trehalose-surfaced dendrimers and at least one agent selected from kinase inhibitors, RTK inhibitors, statins, anti-inflammatory, anti-oxidants, ant-viral, anti-VEGF, angiogenesis inhibitors, antiproliferative inhibitors, apoptosis inhibitors, autophagy inhibitors, trail-agonists, BET inhibitors, Bcr-Abl kinase inhibitors, anti-cancer, mTOR inhibitors, proteosome inhibitors, PARP inhibitors, JAK inhibitors, PPAR-gamma agonists/antagonists, anti-diabetic, galantamine, cabozantinib, AChE-inhibitor, CSDF-1R inhibitors, cannabinoids, ALK kinase inhibitor-1, anaplastic lymphoma kinase (ALK) PROTAC, Beta-2 Adrenergic Receptors: beta-2 adrenergic receptor (ADRB2) agonist, β2-adrenergic receptor blocker, adiponectin receptor (AdipoR) agonist, β3 adrenergic receptor antagonist, β-arrestin/β2-adaptin interaction inhibitor, β-adrenoceptor antagonist. α-adrenergic receptor agonist, muscarinic-3 (M3) agonist. CXCR2 antagonist, CXCR6 antagonist, phosphodiesterase-4 (PDE4) inhibitor, tumor necrosis factor-α (TNF-α) inhibitor, antagonist of H1-histamine receptor, TYK2 inhibitor, TIE-2 inhibitors, 5-HT transporter inhibitor, histamine H3 receptor full antagonist, histamine H1-receptor antagonist, histamine H2-receptor antagonist, histamine H1 and H2 receptor agonist, antihistamine agent, EGFR Inhibitors, PDGFR Tyrosine Kinase Inhibitor III, FAK dual inhibitor, STAT3 Inhibitors, BRD4 inhibitor, RAS inhibitor, glutathione peroxidase 4 (GPX4) inhibitor, c-Myc inhibitor, hCYP1B1 inhibitor, BACE-1 inhibitor, TGF-β receptor kinase inhibitor, ROS1 kinase inhibitor, VEGFR inhibitors, (Vascular Endothelial Growth Factor Receptor), endothelin receptor antagonist, cystic fibrosis transmembrane regulator, ATP-sensitive K+ channel (KATP) inhibitor, TGF-beta/Smad inhibitors, ROCK inhibitors, interleukin-6 (IL-6) receptor antagonist, Toll-like receptor 7 and 8 (TLR7/TLR8) agonist, IRAK4 (Interleukin 1 receptor associated kinase 4) inhibitor, human formyl peptide receptor like-1 (FPRL-1/FPR2) agonist, nuclear factor-kappa B (NF-κB) activators/inhibitors, tumor necrosis factor-α (TNF-α) inhibitor, matrix metalloproteinases (MMP) inhibitor, 5-lipoxygenase (5-LO) inhibitor, antagonist of prostaglandin E2 (PGE2) receptor (EP 1), prostanoid receptor ligand, androgen receptor inhibitor, ROR agonist-1, retinoic acid receptor-related orphan receptor γt (RORγt), IL-17A and inhibits its interaction with the IL-17 receptor, GABA (A) receptors competitive antagonist, GABA aminotransferase activator, Thromboxane receptors antagonists or synthase inhibitors, angiotensin II AT2 receptor agonist, matrix metalloproteinase-2 (MMP-2) selective inhibitor, CGRP receptor activator, antagonist of CGRP receptor, adrenomedullin receptor antagonist, calcium and sodium channel blockers, Beta blockers, Tumor Necrosis Factor-alpha (TNF-alpha) inhibitors (such as Infliximab, Adalimumab, and Etanercept), Interleukin-1: IL-1 inhibitors like Anakinra and Canakinumab, Interleukin-6 (IL-6) Inhibitors like Tocilizumab and Sarilumab, B-cell lymphoma inhibitors, Immunosuppressants, T cell modulator like Efalizumab, IL-23 and IL-12 inhibitors, C5a Receptor agonist, RANKL (Receptor Activator of Nuclear Factor Kappa-B Ligand) inhibitors like Denosumab, Thyroid Hormone Receptor Antagonists, PPARα agonist, Angiotensin Inhibitors, Adenosine A1/A3 Receptor Antagonist, μ-opioid receptor antagonist, antagonist for the κ-opioid receptor, μ opioid receptor partial agonist, kappa opioid receptor (KOR) agonist, competitive antagonist of the NOP receptor, pan-opioid antagonist, δ-opioid receptor (DOR) antagonist, voltage-gated sodium channel (NaV) 1.7 inhibitor, inhibitor of noradrenaline transporter (NET), voltage-gated potassium channel blockers, CGRP receptor activator, antagonist of serotonin receptors, agonist for melatonin receptors, EP3 receptor agonist, EP1- and EP3-receptor agonist, P2X3 receptor (P2X3R) antagonist, inhibitor of P2X3 receptor, purinergic (P2X1) receptor antagonist, selective and non-nucleotide antagonist of P2X3 and P2X2/3 receptors, protease-activated receptor (PAR-2) agonist, orexin receptor antagonists, Tropomyosin-related kinases, COX-3 (Cyclooxygenase-3) inhibitors, diazepam binding inhibitor (DBI) receptor, agonist of benzodiazepine receptor, corticotropin-releasing hormone receptor 1 (CRHR1) antagonist, Inhibitor of β-Secretase and voltage-gated sodium channel, antagonist of metabotropic glutamate receptor type 1 (mGluR1), psychedelics, ligands, dyes, radioligands, siRNA, targeting ligands, bioactive groups, folic acid, polymers, chelating agents (e.g. DOTA, NOTA), or inorganic molecules etc), Oligonucleotides, Suppresses HBV replication, HBV DNA replication inhibitor, HBV capsid assembly modulator, surface antigen (HBsAg) inhibitor, HBV transcriptional suppression, HBV capsid inhibitor, protein assembly modulator, inhibits HBV DNA polymerase, HIV infection inhibitor, HIV infection inhibitor, anti-HBV/HCV/HSV-1/HIV, anti-inflammatory, selective FXR agonist, antiretroviral drug-resistant HIV strains, anti-bacterial agent, Sirtuin 5 deacylase inhibitor, Sirtuin 5 deacylase inhibitor, Sirtuin-1 (SIRT1) activator, DNA methyltransferase 3A (DNMT3A) inhibitor, methyltransferase (DNMT1) selective inhibitor, MGMT (O6-methylguanine DNA methyltransferase) inhibitor, thymidylate synthase inhibitor, G9a/DNA methyltransferases (DNMTs) inhibitor, non-covalent inhibitor for DNA methyltransferase (Dnmt1), inhibiting histone deacetylase and DNA methyltransferase, inhibitor of DNA (cytosine-5)-methyltransferase (Mtase), acetyl-CoA carboxylase (ACC) inhibitor, ACC2 inhibitor, inhibits NLRP3 inflammasome, and is an anti-inflammatory agent, selective connexin 43 (Cx43) hemichannel blocker, connexin 43 (gap junction blocker), targets connexin 26 (Cx26), antibacterial against Gram-positive, Gram-negative and anaerobic organisms, blocks the DNA gyrase and topoisomerase IV activity, activator of peroxisome proliferator-activated receptor-γ coactivator-1α (PGC-1α), Liver kinase B1, AMP-activated protein kinase (AMPK) activator, Cystic fibrosis transmembrane conductance regulator (CFTR), glucagon receptor antagonist, glucagon-like peptide-1 receptor (GLP-1-R) antagonist, dipeptidyl peptidase IV (DPP-IV) inhibitor, cytotoxic tubulin modifier, inhibit catecholamine release, Estrogen receptor antagonist 3, TXNIP inhibitor, GLP-1 receptor agonist 10, neurokinin 2 (NK2) receptor agonist, glucose-dependent insulinotropic polypeptide (GIP) and glucagon-like peptide-1 (GLP-1) receptor agonist, NADH: ubiquinone oxidoreductase inhibitory, G-protein-coupled receptor kinase 5 (GRK5) inhibitor, amylin receptor antagonist, IRE-1α inhibitor, IRE-1α inhibitor, flukicidal agent against liver flukes, Glucocorticoid receptor agonist, melanocortin MC4 receptor antagonist, anti-rabies virus (RABV), melanocortin MC4 receptor antagonist, stimulator of interferon genes (STING) agonists, alpha 1-adrenoceptor blocker, alpha 1-adrenoceptor blocker, inhibits muscarinic acetylcholine receptor (mAChR), μ-opioid receptor antagonist, or potent tubulin polymerization inhibitor,
wherein the at least one agent is located on the surface or interior of the dendrimer and are connected to the dendrimer via a covalent bond or physical entrapment.

In some embodiments, dendrimers have a diameter of 0.1 nm to 1000 nm, more particularly 1 nm to 15 nm.

In some embodiments, dendrimers have a molecular weight of 1000 Daltons to 500,000 Daltons, more particularly 5,000 Daltons to 40,000 Daltons.

### ILLUSTRATIVE EMBODIMENTS

Disclosed herein are several embodiments of 2DG-surfaced dendrimers as described in the numbered clauses below:
1. A dendrimer, comprising
(a) a central core,
(b) one or more branching units, wherein the branching units are
   glucose-based branching units, wherein the glucose may be a mono, di or oligosacchardie, optionally with a linker conjugate thereto; and optionally
(c) one or more therapeutic, prophylactic or diagnostic agents,
   wherein the one or more branching units are conjugated to the central core, and wherein the surface groups of the dendrimer further comprises 2-deoxy-D-glucose (2DG) unit configurations.
2. The glucose dendrimer of clause 1, wherein the dendrimer is configured with the following structure:

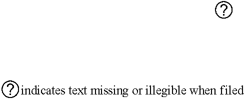

3. A dendrimer composition, comprising: a 2-DG configuration in at least one of an interior, a core, and therebetween the generations from generation 1 to 10, where —OH of 2DG groups are free.
4. A dendrimer composition, comprising: 2DG-dendrimers from generation 1 to having configured surface 2DG molecules, irrespective of their interior and backbone including branching units, linking chemistry, and surface groups.
5. The dendrimer composition of clause 4, wherein the 2DG-dendrimers includes generation 1 (G1) configured as a polyamidoamine bearing hexyne arms, generation 2 (G2) comprises a gallic acid building blocks-based layer, and G3 is configured as a 2DG layer, wherein the dendrimers include a plurality of classical and mixed-layer dendrimers, wherein the 2DG is at the surface and attached on at least one of PAMAM, Polylysine, bisMPA, tetrazine, PPI, PEG-based dendrimers, and glycodendrimers.
6. A composition, comprising: dendrons and hyperbranched polymeric structures with a plurality of 2DG molecules at the surface or interior.
7. A composition, comprising: a 2-DG terminated dendrimer configured around a 2 armed core to 200 armed core.
8. A composition, comprising: a 2-DG terminated dendrimer configured with a size range between 0.1 nm up to 1000 nm.
9. A composition comprising: a 2-DG terminated dendrimer configured by at least one of divergent, convergent, and mixed divergent/convergent routes.
10. A composition, comprising: a 2-DG terminated Janus dendrimer, a hybrid dendrimer and a miktoarm dendrimer, wherein all of the arms are different.
11. A composition, comprising: a plurality of dendrimers linked to at least one of dendrons, linear polymers, hydrogels, and other hyperbranched structures having 2DG molecules at the surface.
12. The composition of clause 9, wherein the dendrimers range from molecular weights of 1000 Da up to 500 kDa, wherein the 2DG molecules are configured on the surface or in combination with one or more other agents.
13. A composition, further comprising: a plurality of mixed layer dendrimers configured with 2DG, wherein the layers are further configured of building blocks comprising 2DG at the surface or anywhere in the interior or both.
14. A composition as in any one of clauses 1-13, further comprising a plurality of PEG linkers varying from 1 PEG unit to 100 PEG units.
15. A composition as in any one of clauses 1-14, further comprising a plurality of hexyne linkers varying from 1 —CH₂ unit to 50 —CH₂, wherein the units further comprise hetero atoms.
16. A composition as in any one of clauses 1-15, further comprising: PEG linkers configured as polymeric or non-polymeric chains as linkers.
17. A composition as in any one of clauses 1-16, further comprising: a plurality of linkages selected from disulfide, ester, ether, carbonate, carbamate, thiol, thioester, cathepsin sensitive, maleimidomethyl, thioether, hydrazine, glucuronide bond, hydrazides, N-alkyl, ethyl, hydroxymethyl, and amide.
18. A composition as in any one of clauses 1-17, further comprising: a plurality of linkers configured between the dendrimers and a therapeutic agent so as to provide a releasable or non-releasable form of the dendrimer-active complexes in vivo, wherein the drug/protein/oligonucleotide releases an active moiety in some cases, where in a plurality of desired dendrimer conjugates, a plurality of therapeutic/bioactive molecules are not released from the dendrimers, wherein for the released active moiety, at least one of disulfide, ester, carbonate, carbamate, thiol, thioester, cathepsin sensitive, hydrazine, glucuronide bond, hydrazides, N-alkyl, ethyl, hydroxymethyl, and amide linkages are present, and wherein for the non-releasable moiety, at least one of ether, thioether and maleimidocaproyl linkages etc are present.
19. A composition as in any one of clauses 1-18, wherein no linker is present in between a drug and a hydroxyl group of 2-deoxy-glucose.
20. A composition as in any one of clauses 1-19, further comprising: a drug, a metal, protein, or a therapeutic molecule delivered by encapsulation/complexation.
21. A composition as in any one of clauses 1-20, further comprising: pharmaceutically accepted salts formed by the addition of any acid or bases.
22. A composition as in any one of clauses 1-21, further comprising: hydrogels, linear polymers, hyperbranched polymers, glycopolymers, biopolymers, implants, or connected to other nano or micro-technologies through cross-linking reactions.
23. The composition of clause 22, wherein the glycopolymers are selected from: hyaluronic acid, chitosan, dextran, and the like.
24. A composition, comprising: a plurality of multifunctional dendrimers with a combination of 2DG with a sugar or a non-sugar group, further including drugs selected from: kinase inhibitors, RTK inhibitors, statins, anti-inflammatory, anti-oxidants, ant-viral, anti-VEGF, angiogenesis inhibitors, antiproliferative inhibitors, apoptosis inhibitors, autophagy inhibitors, trail-agonists, BET inhibitors, Bcr-Abl kinase inhibitors, anti-cancer, mTOR inhibitors, proteosome inhibitors, PARP inhibitors, JAK inhibitors, PPAR-gamma agonists/antagonists, anti-diabetic, galantamine, cabozantinib, AChE-inhibitor, CSDF-1R inhibitors, cannabinoids, ALK kinase inhibitor-1, anaplastic lymphoma kinase (ALK) PROTAC, Beta-2 Adrenergic Receptors: beta-2 adrenergic receptor (ADRB2) agonist, β2-adrenergic receptor blocker, adiponectin receptor (AdipoR) agonist, β3 adrenergic receptor antagonist, β-arrestin/β2-adaptin interaction inhibitor, β-adrenoceptor antagonist. α-adrenergic receptor agonist, muscarinic-3 (M3) agonist. CXCR2 antagonist, CXCR6 antagonist, phosphodiesterase-4 (PDE4) inhibitor, tumor necrosis factor-α (TNF-α) inhibitor, antagonist of H1-histamine receptor, TYK2 inhibitor, TIE-2 inhibitors, 5-HT transporter inhibitor, histamine H3 receptor full antagonist, histamine H1-receptor antagonist, histamine H2-receptor antagonist, histamine H1 and H2 receptor agonist, antihistamine agent, EGFR Inhibitors, PDGFR Tyrosine Kinase Inhibitor III, FAK dual inhibitor, STAT3 Inhibitors, BRD4 inhibitor, RAS inhibitor, glutathione peroxidase 4 (GPX4) inhibitor, c-Myc inhibitor, hCYP1B1 inhibitor, BACE-1 inhibitor, TGF-β receptor kinase inhibitor, ROS1 kinase inhibitor, VEGFR inhibitors, (Vascular Endothelial Growth Factor Receptor), endothelin receptor antagonist, cystic fibrosis transmembrane regulator, ATP-sensitive K+ channel (KATP) inhibitor, TGF-beta/Smad inhibitors, ROCK inhibitors, interleukin-6 (IL-6) receptor antagonist, Toll-like receptor 7 and 8 (TLR7/TLR8) agonist, IRAK4 (Interleukin 1 receptor associated kinase 4) inhibitor, human formyl peptide receptor like-1 (FPRL-1/FPR2) agonist, nuclear factor-kappa B (NF-κB) activators/inhibitors, tumor necrosis factor-α (TNF-α) inhibitor, matrix metalloproteinases (MMP) inhibitor, 5-lipoxygenase (5-LO) inhibitor, antagonist of prostaglandin E2 (PGE2) receptor (EP 1), prostanoid receptor ligand, androgen receptor inhibitor, ROR agonist-1, retinoic acid receptor-related orphan receptor γt (RORγt), IL-17A and inhibits its interaction with the IL-17 receptor, GABA (A) receptors competitive antagonist, GABA aminotransferase activator, Thromboxane receptors antagonists or synthase inhibitors, angiotensin II AT2 receptor agonist, matrix metalloproteinase-2 (MMP-2) selective inhibitor, CGRP receptor activator, antagonist of CGRP receptor, adrenomedullin receptor antagonist, calcium and sodium channel blockers, Beta blockers, Tumor Necrosis Factor-alpha (TNF-alpha) inhibitors (such as Infliximab, Adalimumab, and Etanercept), Interleukin-1: IL-1 inhibitors like Anakinra and Canakinumab, Interleukin-6 (IL-6) Inhibitors like Tocilizumab and Sarilumab, B-cell lymphoma inhibitors, Immunosuppressants, T cell modulator like Efalizumab, IL-23 and IL-12 inhibitors, C5a Receptor agonist, RANKL (Receptor Activator of Nuclear Factor Kappa-B Ligand) inhibitors like Denosumab, Thyroid Hormone Receptor Antagonists, PPARα agonist, Angiotensin Inhibitors, Adenosine A1/A3 Receptor Antagonist, μ-opioid receptor antagonist, antagonist for the κ-opioid receptor, μ opioid receptor partial agonist, kappa opioid receptor (KOR) agonist, competitive antagonist of the NOP receptor, pan-opioid antagonist, δ-opioid receptor (DOR) antagonist, voltage-gated sodium channel (NaV) 1.7 inhibitor, inhibitor of noradrenaline transporter (NET), voltage-gated potassium channel blockers, CGRP receptor activator, antagonist of serotonin receptors, agonist for melatonin receptors, EP3 receptor agonist, EP1- and EP3-receptor agonist, P2X3 receptor (P2X3R) antagonist, inhibitor of P2X3 receptor, purinergic (P2X1) receptor antagonist, selective and non-nucleotide antagonist of P2X3 and P2X2/3 receptors, protease-activated receptor (PAR-2) agonist, orexin receptor antagonists, Tropomyosin-related kinases, COX-3 (Cyclooxygenase-3) inhibitors, diazepam binding inhibitor (DBI) receptor, agonist of benzodiazepine receptor, corticotropin-releasing hormone receptor 1 (CRHR1) antagonist, Inhibitor of β-Secretase and voltage-gated sodium channel, antagonist of metabotropic glutamate receptor type 1 (mGluR1), psychedelics, ligands, dyes, radioligands, siRNA, targeting ligands, bioactive groups, folic acid, polymers, chelating agents (e.g. DOTA, NOTA), or inorganic molecules etc), Oligonucleotides, Suppresses HBV replication, HBV DNA replication inhibitor, HBV capsid assembly modulator, surface antigen (HBsAg) inhibitor, HBV transcriptional suppression, HBV capsid inhibitor, protein assembly modulator, inhibits HBV DNA polymerase, HIV infection inhibitor, HIV infection inhibitor, anti-HBV/HCV/HSV-1/HIV, anti-inflammatory, selective FXR agonist, antiretroviral drug-resistant HIV strains, anti-bacterial agent, Sirtuin 5 deacylase inhibitor, Sirtuin 5 deacylase inhibitor, Sirtuin-1 (SIRT1) activator, DNA methyltransferase 3A (DNMT3A) inhibitor, methyltransferase (DNMT1) selective inhibitor, MGMT (O6-methylguanine DNA methyltransferase) inhibitor, thymidylate synthase inhibitor, G9a/DNA methyltransferases (DNMTs) inhibitor, non-covalent inhibitor for DNA methyltransferase (Dnmt1), inhibiting histone deacetylase and DNA methyltransferase, inhibitor of DNA (cytosine-5)-methyltransferase (Mtase), acetyl-CoA carboxylase (ACC) inhibitor, ACC2 inhibitor, inhibits NLRP3 inflammasome, and is an anti-inflammatory agent, selective connexin 43 (Cx43) hemichannel blocker, connexin 43 (gap junction blocker), targets connexin 26 (Cx26), antibacterial against Gram-positive, Gram-negative and anaerobic organisms, blocks the DNA gyrase and topoisomerase IV activity, activator of peroxisome proliferator-activated receptor-γ coactivator-1α (PGC-1α), Liver kinase B1, AMP-activated protein kinase (AMPK) activator, Cystic fibrosis transmembrane conductance regulator (CFTR), glucagon receptor antagonist, glucagon-like peptide-1 receptor (GLP-1-R) antagonist, dipeptidyl peptidase IV (DPP-IV) inhibitor, cytotoxic tubulin modifier, inhibit catecholamine release, Estrogen receptor antagonist 3, TXNIP inhibitor, GLP-1 receptor agonist 10, neurokinin 2 (NK2) receptor agonist, glucose-dependent insulinotropic polypeptide (GIP) and glucagon-like peptide-1 (GLP-1) receptor agonist, NADH: ubiquinone oxidoreductase inhibitory, G-protein-coupled receptor kinase 5 (GRK5) inhibitor, amylin receptor antagonist, IRE-1α inhibitor, IRE-1α inhibitor, flukicidal agent against liver flukes, Glucocorticoid receptor agonist, melanocortin MC4 receptor antagonist, anti-rabies virus (RABV), melanocortin MC4 receptor antagonist, stimulator of interferon genes (STING) agonists, alpha 1-adrenoceptor blocker, alpha 1-adrenoceptor blocker, inhibits muscarinic acetylcholine receptor (mAChR), μ-opioid receptor antagonist, or potent tubulin polymerization inhibitor.

Disclosed herein are several embodiments of trehalose-surfaced dendrimers as described in the numbered clauses below:
1. A mixed layer dendrimer composition comprising a plurality of layers of building blocks in an interior and a surface layer of one or more trehalose (tre) units. An example embodiment is disclosed of generation 3 dendrimer with 24 trehalose molecules attached to the surface. This is the first example of a mixed layered dendrimer composition having trehalose molecules at the entire peripheral (or surface) layer.
2. A dendrimer composition where trehalose is present in the interior or core or in between the generations, where —OH of trehalose groups are free.
3. A dendrimer from generation 1 to 10 having surface trehalose molecules, irrespective of their interior and backbone including branching units, linking chemistry, and surface groups. In an example embodiment, the trehalose-dendrimer contains G1 as polyamidoamine bearing hexyne arms, G2 as gallic acid building blocks-based layer, and G3 is trehalose layer. Variations include all the classical and mixed-layer dendrimers where trehalose is at the surface whether attached on commercially available ones (PAMAM, Polylysine, bisMPA, tetrazine, PPI, PEG-based dendrimers, or glycodendrimers) or attached on the new dendrimers.
4. A dendronic or hyperbranched polymeric structure with trehalose molecules at the surface or interior.
5. A composition comprising trehalose terminated dendrimer which is constructed around a core with number of arms ranging from 2 to 200.
6. A composition comprising trehalose terminated dendrimer which has the size range between 0.1 nm-1000 nm.
7. A composition comprising trehalose terminated dendrimer which is constructed either by divergent, convergent, and/or mixed divergent/convergent routes and strategies.
8. A composition comprising trehalose terminated Janus dendrimer, hybrid dendrimers and miktoarm dendrimer where all the arms of a dendrimer are different.
9. A dendrimer linked to dendrons, linear polymers, hydrogels, or other hyperbranched structures having trehalose molecules at the surface.
10. A covalent conjugate and/or complex of drugs, imaging agents, targeting ligands, E3 ligase, PROTAC, biologics (siRNA, mRNA, peptides, oligonucleotides, microRNA, genes, antibodies, radioligands, radiotherapeutics, PET agents) with trehalose-dendrimers or dendrons from other claims herein. No restriction on the agents and ligands that can be delivered by conjugation, encapsulation, or complexation on these trehalose dendrimers (for example, oligonucleotides, drugs, peptides, aptamers, antibodies, proteins, RNA/DNA, other biologics).
11. A chemical transformation method to produce compositions, bringing trehalose at the periphery of the dendrimer, through one or more of copper-free strain promoted click, thio-ene click, thiol-yne click, esterification, amidation, Diels-Alder reaction. In the present example, the trehalose is attached via click chemistry on the surface.
12. A dendrimer ranging from molecular weight of 1000 Da to 500 kDa containing trehalose molecules on the surface and/or in combination with other agents.
13. A dendrimer wherein all the layers are made-up of similar building blocks containing trehalose at the surface and/or interior. For example, a dendrimer having trehalose at GO, G1, G2, G3, G4, GS and/or G6 and so on.
14. The dendrimers in clauses 1-13 with PEG linkers to vary from 1 PEG unit to 100 PEG units.
15. The dendrimers in clauses 1-14 with hexyne linkers to vary from 1 —CH₂ unit to 50 —CH₂ units that may also contain hetero atoms.
16. The dendrimers in clauses 1-15 with PEG linkers to have other polymeric or non-polymeric chains as linkers.
17. A therapeutically active drug composition attached with the dendrimers of clauses 1-16 via one or more linkages such as disulfide, ester, ether, carbonate, carbamate, thiol, thioester, cathepsin sensitive, maleimidomethyl, thioether, hydrazine, glucuronide bond, hydrazides, N-alkyl, ethyl, hydroxymethyl, and amide.
18. The dendrimers, compositions, and therapeutics of clauses 1-17, wherein the linkers present are configured to provide a releasable or non-releasable form of the dendrimer-active complexes in vivo that means the drug/protein/oligonucleotide may act as by releasing the active moiety in some cases but in some dendrimer conjugates, these therapeutic/bioactive molecules are not going to be released from the dendrimer and the whole macromolecule is considered as drug. For the releasable constructs, disulfide, ester, carbonate, carbamate, thiol, thioester, cathepsin sensitive, hydrazine, glucuronide bond, hydrazides, N-alkyl, ethyl, hydroxymethyl, and amide linkages are present. For the nonreleasable constructs, ether, thioether and maleimidocaproyl linkages etc are present.
19. The dendrimers of clauses 1-18 wherein no linker is present in between the drugs and the hydroxyl groups of trehalose. Here the complimentary functional groups present on therapeutic, bioactive, ligand, protein, SiRNA, Protac, aptamers, DNA, carbohydrates, oligonucleotides, antibodies are reacted and joined with dendrimer.
20. A drug, metal, protein, or any therapeutic molecule delivered by encapsulation/complexation using the dendrimers of clauses 1-19.
21. A pharmaceutically accepted salt of the dendrimers in clauses 1-20 formed by the addition of one or more acid or base.
22. The dendrimers of clauses 1-21 configured to be used as hydrogels, linear polymers, hyperbranched polymers, glycopolymers (eg hyaluronic acid, chitosan, dextran etc), biopolymers, implants, or connected to other nano or micro-technologies through cross-linking reactions.
23. A multifunctional dendrimer comprised of a combination of trehalose with any other sugars or non-sugar groups including drugs (for example: kinase inhibitors, RTK inhibitors, statins, anti-inflammatory, anti-oxidants, ant-viral, anti-VEGF, angiogenesis inhibitors, antiproliferative inhibitors, apoptosis inhibitors, autophagy inhibitors, trail-agonists, BET inhibitors, Bcr-Abl kinase inhibitors, anti-cancer, mTOR inhibitors, proteosome inhibitors, PARP inhibitors, JAK inhibitors, PPAR-gamma agonists/antagonists, anti-diabetic, galantamine, cabozantinib, AChE-inhibitor, CSDF-1 R inhibitors, cannabinoids, ALK kinase inhibitor-1, anaplastic lymphoma kinase (ALK) PROTAC, Beta-2 Adrenergic Receptors: beta-2 adrenergic receptor (ADRB2) agonist, 132-adrenergic receptor blocker, adiponectin receptor (AdipoR) agonist, 133 adrenergic receptor antagonist, 13-arrestin/132-adaptin interaction inhibitor, 13-adrenoceptor antagonist. a-adrenergic receptor agonist, muscarinic-3 (M3) agonist. CXCR2 antagonist, CXCR6 antagonist, phosphodiesterase-4 (PDE4) inhibitor, tumor necrosis factor-a (TNF-a) inhibitor, antagonist of H1-histamine receptor, TYK2 inhibitor, TIE-2 inhibitors, 5-HT transporter inhibitor, histamine H3 receptor full antagonist, histamine H1-receptor antagonist, histamine H2-receptor antagonist, histamine H1 and H2 receptor agonist, antihistamine agent, EGFR Inhibitors, PDGFR Tyrosine Kinase Inhibitor Ill, FAK dual inhibitor, STAT3 Inhibitors, BRD4 inhibitor, RAS inhibitor, glutathione peroxidase 4 (GPX4) inhibitor, cMyc inhibitor, hCYP1 B1 inhibitor, BACE-1 inhibitor, TGF-13 receptor kinase inhibitor, ROS1 kinase inhibitor, VEG FR inhibitors, (Vascular Endothelial Growth Factor Receptor), endothelin receptor antagonist, cystic fibrosis transmembrane regulator, ATP-sensitive K+ channel (KATP) inhibitor, TGF-beta/Smad inhibitors, ROCK inhibitors, interleukin-6 (IL-6) receptor antagonist, Toll-like receptor 7 and 8 (TLR7/TLR8) agonist, IRAK4 (Interleukin 1 receptor associated kinase 4) inhibitor, human formyl peptide receptor like-1 (FPRL-1/FPR2) agonist, nuclear factor-kappa B (NF-KB) activators/inhibitors, tumor necrosis factor-α (TNF-α) inhibitor, matrix metalloproteinases (MMP) inhibitor, 5-lipoxygenase (5-LO) inhibitor, antagonist of prostaglandin E2 (PGE2) receptor (EP 1), prostanoid receptor ligand, androgen receptor inhibitor, ROR agonist-1, retinoic acid receptor-related orphan receptor yt (RORyt), IL-17A inhibits its interaction with the IL-17 receptor, GABA(A) receptors competitive antagonist, GABA aminotransferase activator, Thromboxane receptors antagonists or synthase inhibitors, angiotensin II AT2 receptor agonist, matrix metalloproteinase-2 (MMP-2) selective inhibitor, CGRP receptor activator, antagonist of CGRP receptor, adrenomedullin receptor antagonist, calcium and sodium channel blockers, Beta blockers, Tumor Necrosis Factor-alpha (TNFalpha) inhibitors (such as Infliximab, Adalimumab, and Etanercept), Interleukin-1: IL-1 inhibitors like Anakinra and Canakinumab, Interleukin-6 (IL-6) Inhibitors like Tocilizumab and Sarilumab, B-cell lymphoma inhibitors, Immunosuppressants, T cell modulator like Efalizumab, IL-23 and IL-12 inhibitors, C5a Receptor agonist, RANKL (Receptor Activator of Nuclear Factor Kappa-B Ligand) inhibitors like Denosumab, Thyroid Hormone Receptor Antagonists, PPARa agonist, Angiotensin Inhibitors, Adenosine A1/A3 Receptor Antagonist, μ-opioid receptor antagonist, antagonist for the K-opioid receptor, μ opioid receptor partial agonist, kappa opioid receptor (KOR) agonist, competitive antagonist of the NOP receptor, pan-opioid antagonist, a-opioid receptor (DOR) antagonist, voltage-gated sodium channel (NaV) 1.7 inhibitor, inhibitor of noradrenaline transporter (NET), voltage-gated potassium channel blockers, CGRP receptor activator, antagonist of serotonin receptors, agonist for melatonin receptors, EP3 receptor agonist, EP1- and EP3-receptor agonist, P2X3 receptor (P2X3R) antagonist, inhibitor of P2X3 receptor, purinergic (P2X1) receptor antagonist, selective and non-nucleotide antagonist of P2X3 and P2X2/3 receptors, protease-activated receptor (PAR-2) agonist, orexin receptor antagonists, Tropomyosin-related kinases, COX-3 (Cyclooxygenase-3) inhibitors, diazepam binding inhibitor (DBI) receptor, agonist of benzodiazepine receptor, corticotropin-releasing hormone receptor 1 (CRHR1) antagonist, Inhibitor of 13-Secretase and voltage-gated sodium channel, antagonist of metabotropic glutamate receptor type 1 (mGluR1), psychedelics, ligands, dyes, radioligands, siRNA, targeting ligands, bioactive groups, folic acid, polymers, chelating agents (e.g. DOTA, NOTA), or inorganic molecules etc), Oligonucleotides, Suppresses HBV replication, HBV DNA replication inhibitor, HBV capsid assembly modulator, surface antigen (HBsAg) inhibitor, HBV transcriptional suppression, HBV capsid inhibitor, protein assembly modulator, inhibits HBV DNA polymerase, HIV infection inhibitor, HIV infection inhibitor, antiHBV/HCV/HSV-1/HIV, anti-inflammatory, selective FXR agonist, antiretroviral drug resistant HIV strains, anti-bacterial agent, Sirtuin 5 deacylase inhibitor, Sirtuin 5 deacylase inhibitor, Sirtuin-1 (SIRT1) activator, DNA methyltransferase 3A (DNMT3A) inhibitor, methyltransferase (DNMT1) selective inhibitor, MGMT (O6-methylguanine DNA methyltransferase) inhibitor, thymidylate G9a/DNA methyltransferases (DNMTs) inhibitor, synthase non-covalent inhibitor, inhibitor for DNA methyltransferase (Dnmt1), inhibiting histone deacetylase and DNA methyltransferase, inhibitor of DNA (cytosine-5)-methyltransferase (Mtase), acetyl-CoA carboxylase (ACC) inhibitor, ACC2 inhibitor, inhibits NLRP3 inflammasome, and is an anti-inflammatory agent, selective connexin 43 (Cx43) hemichannel blocker, connexin 43 (gap junction blocker), targets connexin 26 (Cx26), antibacterial against Gram-positive, Gram-negative and anaerobic organisms, blocks the DNA gyrase and topoisomerase IV activity, activator of peroxisome proliferatoractivated receptor-y coactivator-1a (PGC-1a), Liver kinase B1, AMPactivated protein kinase (AMPK) activator, Cystic fibrosis transmembrane conductance regulator (CFTR), glucagon receptor antagonist, glucagon-like peptide-1 receptor (GLP-1-R) antagonist, dipeptidyl peptidase IV (OPP-IV) inhibitor, cytotoxic tubulin modifier, inhibit catecholamine release, Estrogen receptor antagonist 3, TXNIP inhibitor, GLP-1 receptor agonist 10, neurokinin 2 (NK2) receptor agonist, glucose-dependent insulinotropic polypeptide (GIP) and glucagon-like peptide-1 (GLP-1) receptor agonist, NADH: ubiquinone oxidoreductase inhibitory, G-protein-coupled receptor kinase 5 (GRKS) inhibitor, amyl in receptor antagonist, IRE-1 a inhibitor, IRE-1 a inhibitor, flukicidal agent against liver flukes, Glucocorticoid receptor agonist, melanocortin MC4 receptor antagonist, anti-rabies virus (RABV), melanocortin MC4 receptor antagonist, stimulator of interferon genes (STING) agonists, alpha 1-adrenoceptor blocker, alpha 1-adrenoceptor blocker, inhibits muscarinic acetylcholine receptor (mAChR), μ-opioid receptor antagonist, or potent tubulin polymerization inhibitor.

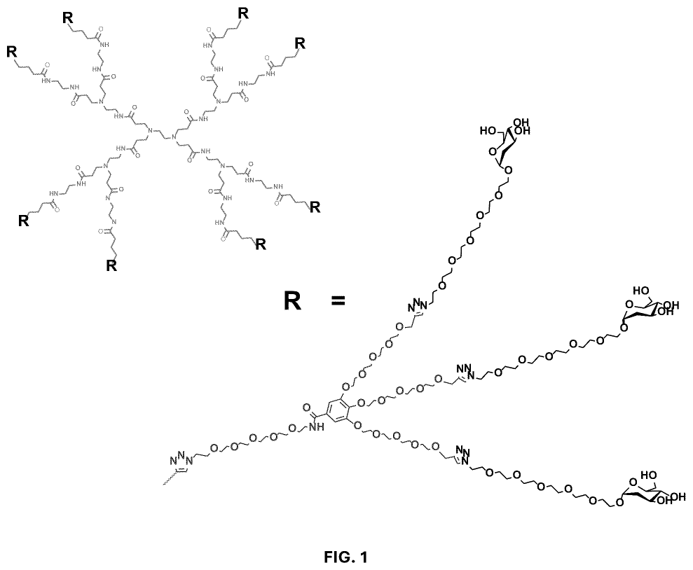

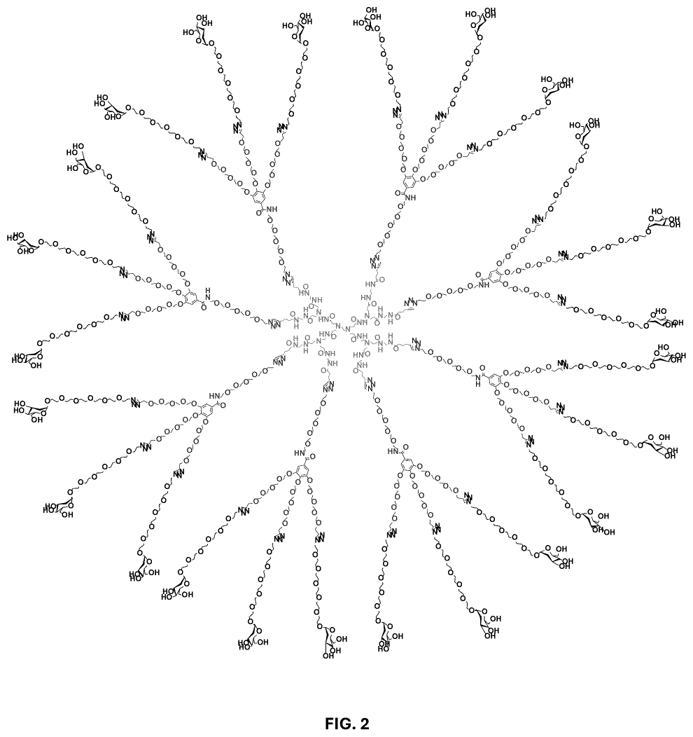

Turning to the drawings, FIG. 1 and FIG. 2 illustrate the structure of a novel dendrimer composition, as disclosed herein, having a mixed layers interior and 2-deoxyglucose molecules (2DG) on the surface. The 2DG-dendrimer is a mixed layer dendrimer having 24 2-DG molecules as part of the peripheral layer. The dendrimer is comprised of biocompatible building blocks and GRAS reagents (˜17 wt % 2DG, ˜57 wt % PEG, ˜13 wt % triazole, ˜9 wt % core, ˜4 wt % gallic acid). The 2DG-dendrimer is synthesized using convergent synthetic route where the mixed layer dendrons are first constructed and then clicked on the multi-arm core.

### EXAMPLES

### 1. Chemistry-Experimental Section

### 1.1 Materials

All starting materials and reagents were purchased from Sigma-Aldrich and Merck. Commercially available reagents and solvents for synthesis were analytical grade and used without further purification. Thin-layer chromatography was performed on a film of silica gel that contained a fluorescent indicator F₂₅₄ supported on an aluminum sheet (Merck). Spectra/Por dialysis membranes were purchased from Repligen.

### 1.2 Instrumentation

Nuclear Magnetic Resonance (NMR) spectra of samples were recorded on a Bruker spectrometer operating at 500 MHz at 25° C. The samples were prepared in deuterated chloroform (CDCl₃), deuterated DMSO (DMSO-d₆) or deuterated water (D₂O).

Microwave reactions were performed in a Biotage Initiator+ instrument using a sealed 10 mL/20 mL process vials. Reaction times refer to irradiation time at the target temperature, not the total irradiation time. The temperature was measured with an IR sensor.

The purity and drug release studies were analyzed using high-performance liquid chromatography (HPLC). The HPLC was performed using a Waters Acquity Arc system (Milford, MA, USA), equipped with binary pumps, 2998 PDA detector, and a 2475 fluorescence detector. The analyses were performed using Waters Empower software. The samples were run using Waters C18 symmetry 300, 5 μm, 4.6×250 mm column using a gradient flow method starting with 90:10 (Solvent A: 0.1% TFA and 5% ACN in water; Solvent B: 0.1% TFA in ACN), gradually increasing to 50:50 (A:B) at 20 minutes, 10:90 (A:B) at 38 minutes finally returning to 90:10 (A:B) at 40 minutes. A flow rate of 1 mL/min was maintained during the run. The dendrimers and drug conjugates were detected at 210 and 254 nm. The 2DG-D-Cy5 was detected at 650 nm. The particle size and zeta potential distribution were determined by dynamic light scattering (DLS) using a Malvern Zetasizer Nano 90 (Westborough, MA). To measure the size, the 2DG-D was dissolved in deionized water (18.2Ω) to create a solution with a final concentration of 0.5 mg/mL. This solution was then passed through 0.2 μm syringe filters (Pall Corporation, 0.2 μm HT Tuffryn membrane) directly into the cell (UV transparent disposable cuvette, dimensions: 12.5×12.5×45 mm). For zeta potential measurement, a sample at a concentration of 0.2 mg/mL in 10 mM NaCl was prepared using the same procedure. The measurements were conducted using a Malvern Zetasizer Nanoseries disposable folded capillary cell.

High resolution mass spectra were performed by Dr. Yue Li in the department of Chemistry and Biochemistry at the University of Maryland-College Park using ESI by direct infusion on a Bruker MALDI-TOF (trans-2-[3-(4-tert-Butylphenyl)-2-methyl-2-propenylidene]malononitrile matrix), and Bruker Q-TOF.

### 1.3 Synthesis Schemes

### Synthesis of 2DG-D

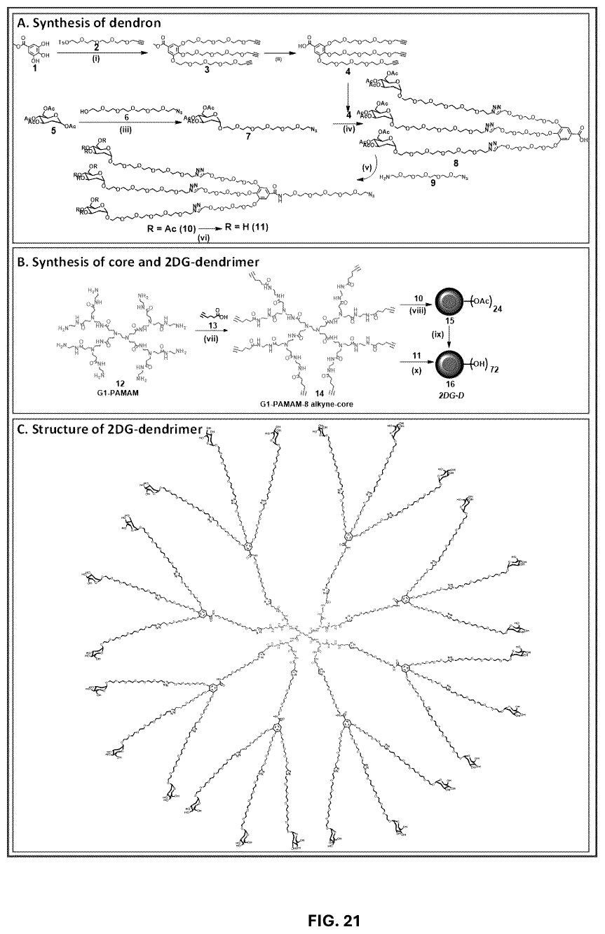

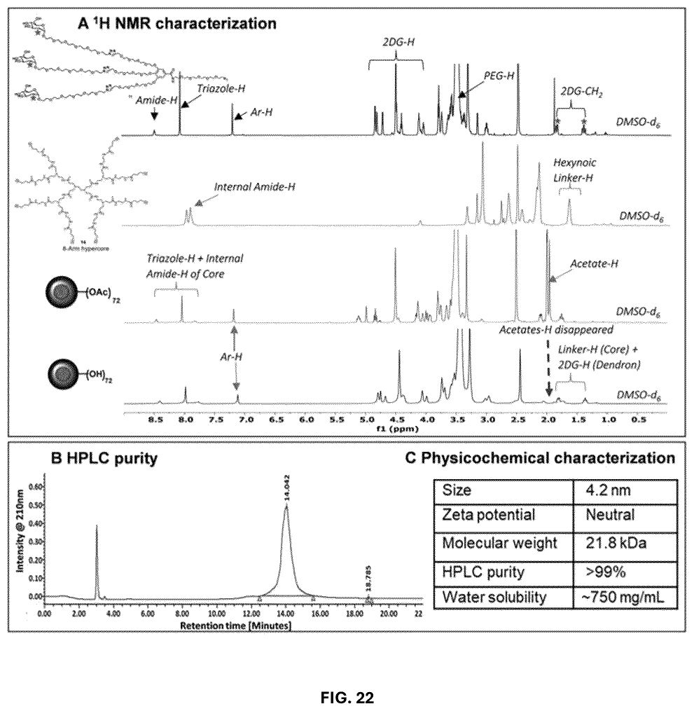

The 2DG-D synthesis was initiated with the preparation of a clickable G2 dendron (FIG. 21A). Methyl gallate (1) was alkylated at three hydroxyls with propargyltetraethyleneglycol-4-methylbenzenesulfonate (2) to afford tri-alkynated compound (3). The ¹H NMR analysis clearly showed the characteristic alkyne signals (3H) at δ 2.43 ppm. This was followed by hydrolysis with lithium hydroxide to produce compound (4) in 80% overall yield. On the other hand, peracetylated 2-deoxy-D-glucose (5) was modified with a short PEG linker with azide focal point. This involved the glycosylation of 2DG with azido-HEG-alcohol (6) in presence of Lewis acid BF₃·OEt₃, resulting in compound (7) as α-anomer (α_{D}²⁰=53.8° (c=1, CHCl₃). The resultant compound (7) was then clicked to the three acetylene branches of gallic acid monomer (4) via CuAAC reaction under microwave irradiations to yield compound (8). The disappearance of acetylene protons and the appearance of triazole (3H) protons at δ 7.69-7.75 ppm confirmed the completion of click reaction. Furthermore, the proton NMR spectrum also revealed the presence of acetate protons (27H) linked to three 2DG sugars, exhibiting chemical shifts within the range of δ 1.96-2.03 ppm. The carboxylic acid group on compound (8) was then reacted with azido-PEG-5-amine (9) using EDC-HOBt promoted amide coupling to afford acetyl-protected dendron (10). Appearance of characteristic amide proton (NH) at δ 8.46 ppm as well as additional PEG protons in the NMR confirmed the structure of compound 10. Subsequently, de-O-acetylation of protected dendron (10) under Zemplén transesterification conditions (NaOMe, MeOH) provided the final deprotected 2DG azide dendron (11) in quantitative yield (FIG. 21A). The ¹H NMR clearly showed the disappearance of acetate protons (FIG. 22A).

Next, we synthesized the core based on PAMAM generation-1 amine dendrimer (12) which carried 8 amine functional groups on the periphery. It was reacted with the 5-hexynoic acid (13) in the presence of amide coupling reagents to produce 8-armed acetylene functionalized core (14) in 80% yield (FIG. 21B). The complete per-NH₂-acetylene insertion at all eight terminals was confirmed by ¹H NMR analysis, which showed the characteristic methylene (—CH₂) protons (16H) of attached hexynoic acid linker at δ 1.62-1.69 ppm and additional new amide protons (8-NH—) along with internal amides (12-NH—) of G1 PAMAM dendrimer at 7.61-8.21 ppm (FIG. 22A).

Finally, the synthesis of the 2DG-D was carried out through two separate synthetic methods, one involving protected 2DG-G2 azide dendron (10) and the other using unprotected 2DG-G2 azide dendron (11) as depicted in FIG. 21B. To construct the dendrimer via the protected route, the CuAAC click reaction was performed between the octa-alkyne G1 PAMAM dendrimer core (14) and the peracetylated 2DG-azide (10) using classical click reagents, a catalytic amount of copper sulfate pentahydrate (5 mol % per alkyne), and sodium ascorbate (10 mol % per alkyne). The reaction was carried out under microwave (MW) irradiation at 40° C. for 10 h to achieve 2DG-OAc dendrimer 15 (FIG. 21B) in excellent yield. The appearance of new triazole (8H) protons at δ7.7 ppm confirmed the completion of click. In addition, the number of acetate protons (216H) at δ 1.93-2.01 ppm corresponds to the attachment of 24-2DG units further confirming the desired structure (FIG. 22A). The deprotection of acetate groups was achieved via Zemplén transesterification (NaOMe in methanol) to yield the final dendrimer 2DG-D (16) with 72-OH in 95% yield. The disappearance of acetate peaks in the proton NMR confirmed the formation of the deprotected product (FIG. 22A). We used protected route to confirm the successful participation of all acetylene branches in the click reaction through the presence of acetate protons in the NMR which made characterization easier and allowed us to observe any defects or missing arms in the final dendrimer. Within the ¹H NMR spectra, the acetyl protons associated with eight dendrons of 2DG were evident as separate, non-overlapping peaks, distinct from the other protons present in the macromolecule. These characteristic peaks offered a precise way to confirm the successful completion of the reaction. The same deprotected dendrimer 16 was synthesized by the reaction of PAMAM core (14) with unprotected 2DG-azide dendron (11) via click reaction at 80° C. under MW irradiation in a 95% yield. The 2DG-D was purified through tangential flow filtration (TFF) utilizing a 3 kDa cassette. As per the HPLC data, the final dendrimer displayed a purity level of >99% (FIG. 22B).

The complete structure of 2DG-D is shown in FIG. 21C. The physicochemical properties of 2DG-D are presented in FIG. 22C. The 2DG-D exhibits high water solubility (750 mg/mL) which limits the need of excipients for systemic formulations. The size of 2DG-D is 4.2 nm which allows the clearance from off-target organs through renal filtration. The 2DG-D exhibits neutral zeta potential which is important for the movement of dendrimer within the brain tissue parenchyma. Both the protected and unprotected synthetic approaches resulted in the production of 2DG-D with excellent yields, high purity, and consistent reproducibility. All intermediates and the final dendrimer were characterized via NMR and mass spectroscopy. The synthetic approach employed here doesn't necessitate the use of excessive chemicals or reagents as is often required in traditional dendrimer synthesis. Commercially available and most widely used PAMAM dendrimers are typically manufactured using large amounts of reactants, which still frequently results in structural defects at higher generations due to sluggishness of the chemical reactions at multiple ends. On the contrary, this entire synthetic process presented here is green, reproducible, and cost-effective, making it well-suited for commercial applications.

### Reproducibility of 2DG-D Synthesis

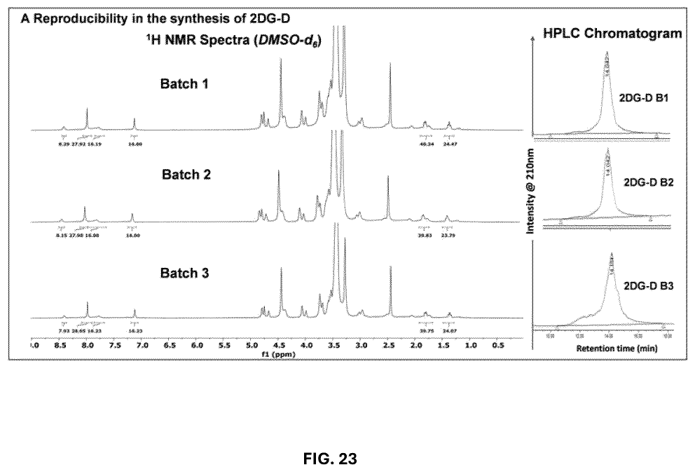

The major hurdle in the clinical translation of nanoparticle-based therapeutics is the lack of reproducibility and scalability of their synthesis process. To address these issues, we synthesized 2DG-D using a simple, convenient, and expedited synthetic strategy that allowed the precise characterization of intermediates at each step as demonstrated above. To further validate the reproducibility in the synthesis of 2DG-D, we constructed several 5 g-scale batches of 2DG-D and compared their ¹H NMRs and purity by HPLC. ¹H NMR spectra of three different batches (FIG. 23A, left) clearly depicted the presence of 24-2DG molecules on the surface, showing consistency in the synthesis of 2DG-D. The HPLC chromatogram of these three batches of 2DG-D showed corresponding peaks at same retention time (14 minutes), confirming the consistency in the purity of these batches (FIG. 23A, right).

### Results and Discussion (Divergent Synthesis of 2DG-D)

Although the convergent approach is a robust method for the synthesis of 2DG-D allowing us to reproducibly construct several gram scale batches of 2DG-D, there is still a room for improvement. In our convergent synthesis approach, we clicked a bulky dendron (2 kDa) with a generation 1 (G1) acetylene-terminated poly(amidoamine) (PAMAM) dendrimer core, which necessitated extended purification processes due to the use of dendron in excess along with its substantial size. Recognizing this as a prospective technical obstacle for generating substantial quantities of highly pure 2DG-D, we here designed and developed an alternate divergent route.

The introduction of smaller building blocks at each step of the divergent synthesis approach makes purification easier and more efficient. Furthermore, the divergent pathway provides additional avenues for constructing libraries of different dendrimers along the way.

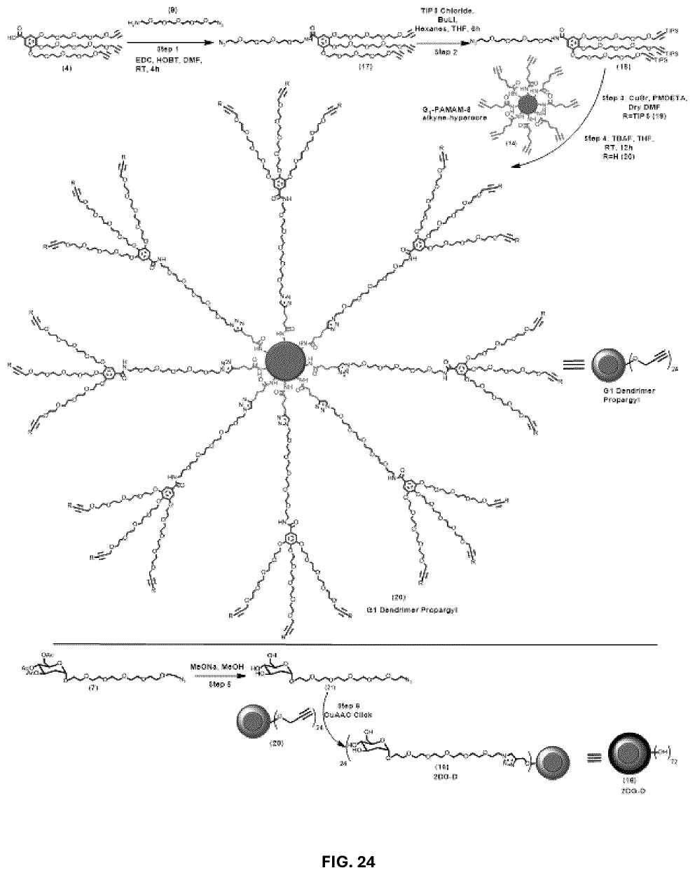

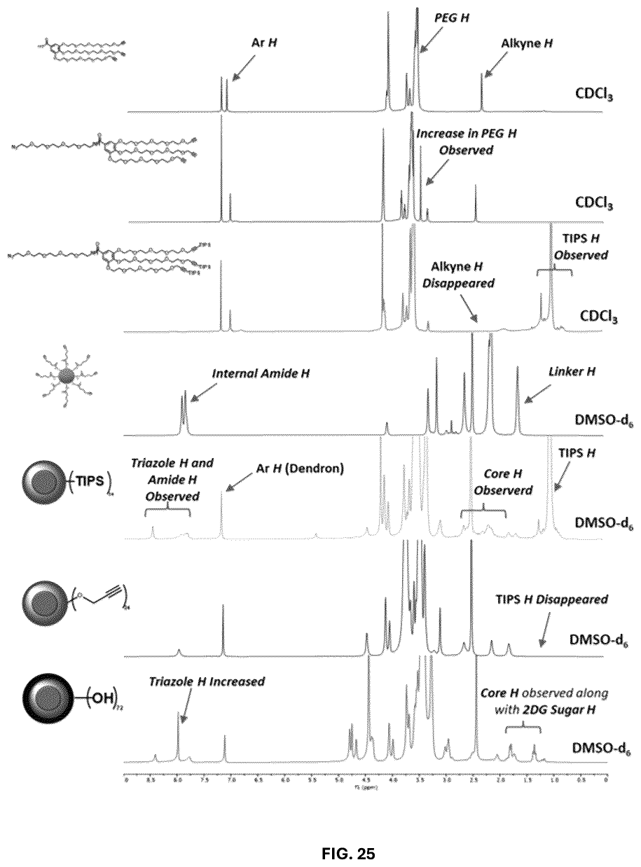

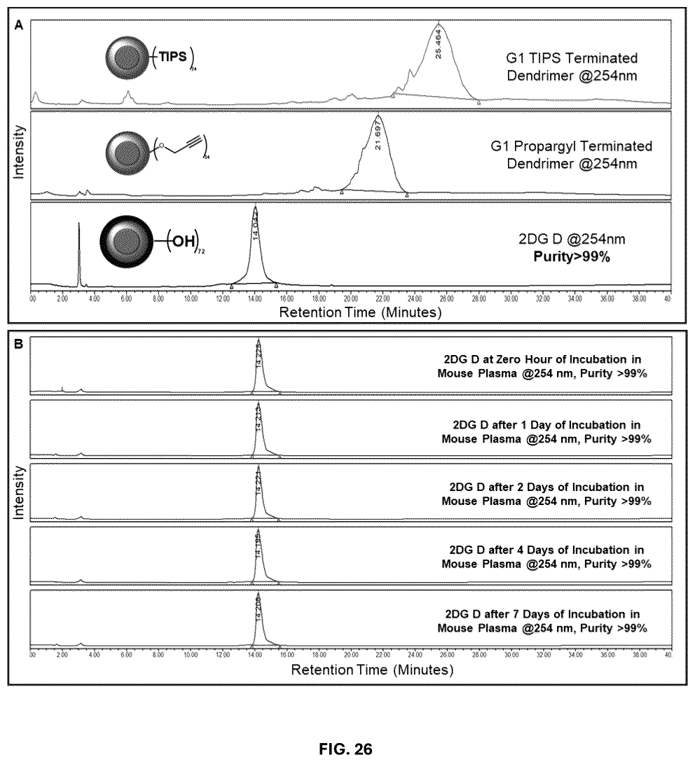

The divergent synthesis of 2DG-D began with the preparation of gallic acid-based tri-alkynated compound (4). FIG. 24. The carboxylic acid group of compound (4) was subsequently conjugated with azido-PEG-5-amine (9) through EDC-HOBt promoted amide coupling, resulting in the formation of compound (17). The ¹H NMR analysis showed the characteristic amide signal at δ 8.51 ppm along with an increase in PEG signals between δ 3.21 and δ 3.81 ppm (FIG. 25). Next, we protected the alkynes with tri-isopropylsilane (TIPS) groups to avoid self-coupling in click reaction. Initially, we encountered challenges in this reaction when employing 1,8-Diazabicyclo [5.4.0] undec-7-ene (DBU) as the base. However, the utilization of n-butyl lithium proved effective in deprotonation of the alkynes, finally resulting in the formation of compound 18. The disappearance of acetylene protons and appearance of TIPS protons at δ 1.00 and δ 1.19 ppm in the NMR confirmed the formation of compound 18 (FIG. 25). This was followed by the conjugation of the TIPS-protected compound 18 to the G1 PAMAM 8-alkyne hypercore (14) via CuAAC click chemistry. The appearance of triazole (8H) protons at δ7.95 ppm alongside the aromatic (δ7.19 ppm) and amide protons (δ8.46-δ8.48 ppm) from the dendrons and the internal amide protons from the core (δ7.82-δ7.86 ppm) confirmed the completion of click. In addition, the number of TIPS protons (504H) at δ 0.94-1.24 ppm corresponded to the 24 TIPS groups further confirming the desired structure (FIG. 25). The HPLC chromatogram of the compound at 25.45 minutes displayed a purity of >97% (FIG. 26A). After this, we removed the TIPS group using TBAF, resulting in the formation of the G1 propargyl-terminated dendrimer (19) (FIG. 24). The disappearance of TIPS protons confirmed the formation of compound 19 (FIG. 24). The HPLC chromatogram showed a shift in retention time from 25.5 minutes for TIPS terminating dendrimer (18) to 21.7 minutes for the propargyl terminating dendrimer (19) (FIG. 26A). At this stage of the synthetic process, the dendrimer, featuring 24 alkyne surface groups, can be used for the conjugation of diverse ligands or various sugar heads modified with an azide linker through CuAAC click reactions. This enables the creation of libraries of dendrimers tailored for various medical applications. Next, we synthesized 2DG-PEG-azide (21) by de-acetylating per-acetylated 2DG-PEG-azide (7) under Zemplén transesterification conditions (NaOMe, MeOH) in quantitative yield (FIG. 24). The ¹H NMR clearly showed the disappearance of acetate protons. Finally, the synthesis of the 2DG-D (16) was achieved through the CuAAC reaction between the 24-alkyne armed G1 dendrimer (19) and the 2DG-azide (21) using classical click reagents, a catalytic amount of copper sulfate pentahydrate (5 mol % per alkyne), and sodium ascorbate (10 mol % per alkyne) in 83% yield. The appearance of new triazole (24H) protons at δ8.04 ppm confirmed the completion of click. In addition, the appearance of protons from the second carbon (C2) of sugar (48H) at δ 1.43-δ 1.85 ppm corresponded to the attachment of 24-2DG units further confirming the desired structure with 72-OH (FIG. 25). The 2DG-D was easily purified through a 12-hour dialysis utilizing a 1 kDa membrane, overcoming the challenge of extended purification durations encountered in the convergent approach. The HPLC data showed a shift in retention time from 21.7 minutes for the propargyl terminating dendrimer (19) to 14 minutes for the 2DG-D (16), with a purity level of >99% of the final dendrimer (FIG. 26A). The detailed characterization of intermediates and final dendrimer are presented in the supplementary information.

We assessed the dendrimer's stability under physiological conditions. The 2DG-D was subjected to incubation in mouse plasma at a concentration of 1 mg/mL, maintained at 37° C. for 7 days. HPLC chromatograms obtained at different time intervals consistently indicated a purity level exceeding 99% (FIG. 26B), showing the dendrimer's stability throughout the duration of the experiment.

### Synthesis of Tre-D

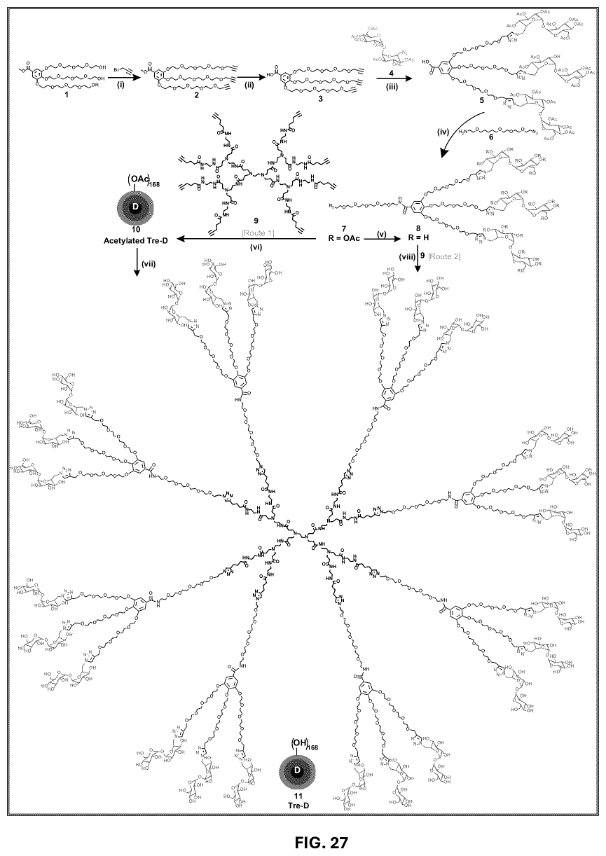

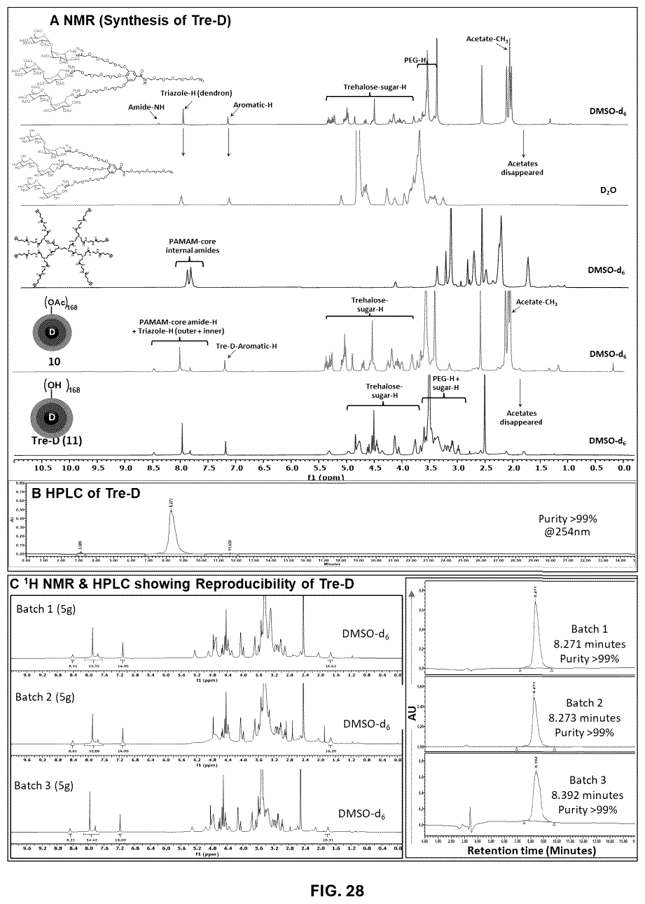

The synthesis of Tre-D begins with the preparation of a clickable generation-2 (G2) dendron 26, as shown in FIG. 27. The process initiates with the propargylation of tri-hydroxyl gallate (22) using sodium hydride in anhydrous DMF, yielding compound 3. The successful formation of compound 3 is confirmed by ¹H NMR analysis, which displays characteristic alkyne signals (3H) at δ 2.43 ppm. In the next step, the hydrolysis of the methyl ester in compound 3 μsing lithium hydroxide in a water: THE mixture affords compound 4 with a 78% yield. Next, the tri-propargylated gallic acid monomer 4 undergoes a CuAAC reaction with peracetylated trehalose azide 23 in DMF at 50° C. for 12 h to afford compound 24. The disappearance of acetylene protons at δ 2.31 ppm and the appearance of triazole protons (3H) at δ 8.00 ppm confirmed the completion of the click reaction. The ¹H NMR spectrum also reveals acetate protons (63H) corresponding to the three trehalose sugars, appearing at δ 1.95-2.05 ppm. Following this, the carboxylic acid group of the compound 24 is reacted with azido-PEG-5-amine 9 μsing EDC-HOBt amide coupling, affording acetate protected dendron 25 in 85% yield. The formation of compound 25 is confirmed by the appearance of a characteristic amide proton at δ 8.46 ppm, along with signals from the PEG protons in the ¹H NMR spectrum (FIG. 28). De-O-acetylation of dendron 25 is then performed under Zemplén transesterification conditions (using NaOMe in methanol), yielding the final azide-terminating deprotected trehalose dendron 26 in quantitative yield. The successful completion of the reaction is confirmed by the disappearance of acetate protons in the δ 1.95-2.04 ppm region (FIG. 28). Next, we synthesized the 8-armed alkyne-functionalized G-1 PAMAM core (14).

We synthesized Tre-D using two synthetic routes. We first opted to perform the reaction using acetate-protected trehalose azide dendron (25) (Route 1, FIG. 27). In the protected route, a CuAAC reaction was performed between the octa-alkyne G-1 PAMAM dendrimer core (14) and the acetate-protected trehalose azide dendron (25). Conventional CuAAC conditions were employed where a catalytic amount of copper sulfate pentahydrate (5 mol % per alkyne) and sodium ascorbate (10 mol % per alkyne) was used, and the reaction was conducted under microwave irradiation at 40° C. for 10 h. This method yielded acetate-protected Tre-D (27) in quantitative yield. The structure of the product was confirmed by the presence of additional triazole protons at δ 7.93 ppm and acetate protons at δ 1.86-2.00 ppm, corresponding to the attachment of 24 trehalose units (FIGS. 27 and 28A). This approach was particularly advantageous in characterizing the dendrimer, as the huge peaks from acetate protons occupy a region in the ¹H NMR spectrum that avoids signal interference. Deprotection of the acetate groups was carried out through Zemplén transesterification using sodium methoxide (NaOMe) in methanol, which yielded the final Tre-D (28) with 168 hydroxyl groups in 90% yield. The disappearance of the acetate peaks in the proton NMR spectrum confirmed the successful formation of the deprotected product (FIGS. 27 and 28A).

For route 2, we performed the CuAAC reaction between the core (14) and the deprotected trehalose-azide dendron (26) under similar conditions (Route 2, FIG. 27), leading to the formation of the final Tre-D (28). Both synthetic routes using protected and deprotected dendrons consistently produced excellent and reproducible results. Purification of the final Tre-D was accomplished through dialysis using a 3 kDa membrane. HPLC analysis of the dendrimer (28) confirmed a purity exceeding 99% (FIG. 28B). The characterization of Tre-D and its intermediates was conducted using a range of techniques, including ¹H NMR, ¹³C NMR, HPLC, MALDI-TOF, and HRMS. As shown in FIG. 28A, the ¹H NMR spectra display the appearance and disappearance of characteristic peaks corresponding to various intermediates, which simplify the interpretation of the dendrimer's structure. Tre-D demonstrates exceptional water solubility (>1 g/mL), which makes it a promising nanocarrier for hydrophobic drugs, hence eliminating the need for additional excipients for final drug products.

### Reproducibility of Tre-D Synthesis

One of the primary challenges in translating nano-therapeutics into clinical applications is developing synthesis processes that are both reproducible and scalable. To overcome the batch-to-batch related heterogeneity associated with macromolecular synthesis, we kept the design of Tre-D very simple and straightforward, and stitched the building blocks together with highly facile and efficient chemical transformation, CuAAC. To evaluate the reproducibility of Tre-D synthesis, we produced multiple 5-gram scale batches of Tre-D and compared their ¹H NMR spectra along with purity via HPLC analysis. The ¹H NMR spectra from three independent batches (FIG. 28C) consistently confirmed the presence of 24 trehalose dendrons on the surface, indicating the formation of defect-free dendrimer. Additionally, the HPLC chromatogram of each batch revealed peaks at the same retention time (8.2 minutes), further confirming batch-to-batch purity (FIG. 28C). The high and consistent yields across batches confirm the robustness of this synthetic method, suggesting its scalability for potential clinical translation.

### 1.4 Synthesis Protocols

### Synthesis of Intermediates and Dendrimers

### 1. 2 Deoxy Glucose Dendrimer

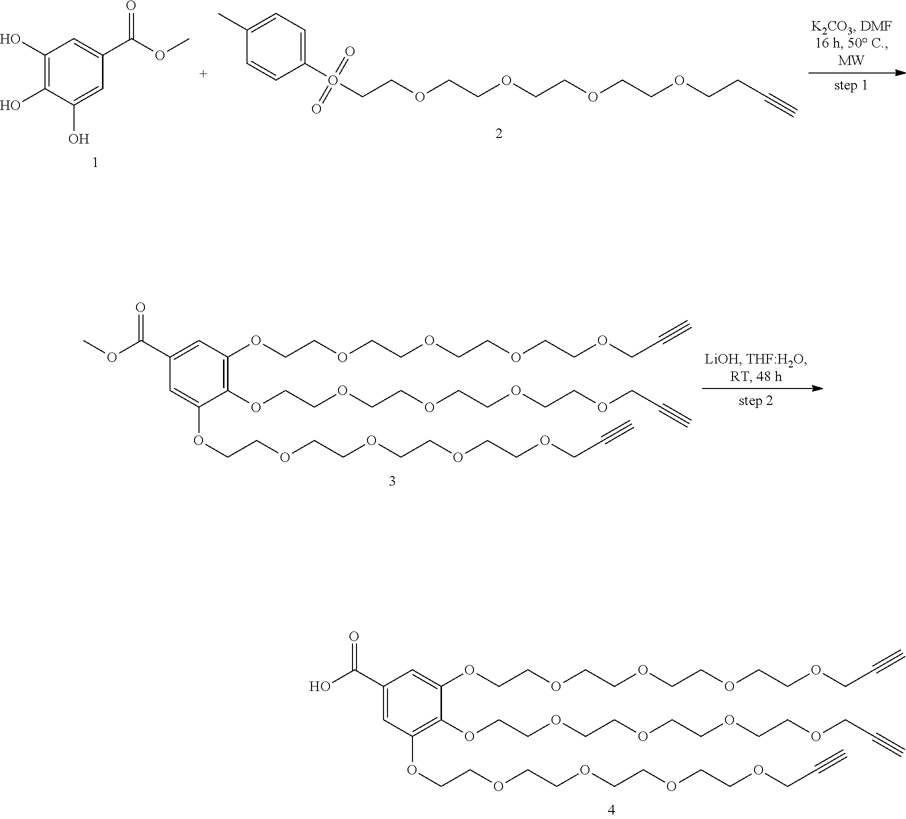

Synthesis of methyl 3,4,5-tris((3,6,9,12-tetraoxapentadec-14-yn-1-yl)oxy)benzoate (3): Compound 1 (600 mg, 1.0 eq, 3.26 mmol) and K₂CO₃ (3.6 g, 26 mmol, 8.0 eq) was dissolved in a 5 mL of DMF in a microwave glass vial under inert atmosphere. After 10 minutes, solution of Compound 2 (5g, 4.0 eq, 13 mmol) in 7.5 mL DMF was added dropwise to the mixture. The reaction vial was irradiated to 50° C. for 16 hours in microwave synthesizer. After completion of the reaction as evident from TLC, DCM was added to reaction mixture and filtered to remove K₂CO₃. Filtrate was washed with water (3×50 mL) and chilled brine (2×50 ml), dried over Na₂SO₄, and evaporated in vacuo. The crude product was purified by silica flash column chromatography [methanol/dichloromethane, 4:96 (v/v)] to afford compound 3 in 60% yield.

Brown viscous liquid; ¹H NMR (500 MHz, CDCl₃) δ 2.43 (s, 3H), 3.63-3.72 (m, 35H), 3.78 (t, J=5.2 Hz, 3H), 3.84-3.90 (m, 7H), 4.17-4.25 (m, 12H), 7.29 (s, 2H). ¹³C NMR (125 MHz, DMSO-d₆) δ 52.65, 57.94, 60.67, 68.97, 68.99, 69.39, 69.96, 70.22, 70.25, 70.30, 70.32, 70.38, 70.41, 72.40, 72.81, 77.55, 77.59, 80.79, 108.54, 124.82, 142.35, 152.48, 166.28. (MALDI-TOF) m/z: calculated for C₄₁H₆₂O₁₇ [M+Na]⁺: 849.3885; found 849.3870.

Synthesis of 3,4,5-tris(2-(2-(2-(2-hydroxyethoxy)ethoxy)ethoxy)ethoxy)benzoic acid (4): To stirred solution of Compound 3 (1 g, 1.0 eq, 1.21 mmol) in 5 mL THF:H₂O (4:1) was added solution of LiOH·H₂O (290 mg, 10 eq, 12.1 mmol) 1 mL DI water. The reaction mixture was stirred at room temperature for 48 h until complete conversion was achieved. The reaction mixture was diluted with water (150 mL), acidified (pH 2-4) with dropwise addition of 1N HCl and extracted with DCM (2×100 mL). The combined organic layer was washed brine (50 mL), then dried (Na₂SO₄), filtered, and evaporated in vacuo. The crude product was purified by silica flash column chromatography [methanol/dichloromethane, 6:94 (v/v)] to afford compound 4 in 95% yield.

Brown viscous liquid; ¹H NMR (500 MHz, CDCl₃) δ 2.31 (m, 3H), 3.49-3.59 (m, 35H), 3.67 (t, J=5.1 Hz, 3H), 3.73 (t, J=5.1 Hz, 4H), 4.05-4.14 (m, 12H), 7.14 (s, 2H). ¹³C NMR (125 MHz, CDCl₃) δ 58.39, 58.41, 68.93, 69.09, 69.10, 69.70, 70.38, 70.40, 70.55, 70.58, 70.59, 70.63, 70.67, 70.68, 70.70, 70.86, 72.46, 74.61, 74.65, 79.63, 79.68, 109.74, 124.18, 143.20, 152.33, 169.93. (MALDI-TOF) m/z: calculated for C₄₀H₆₀O₁₇ [M−H]⁺: 811.3752; found 811.3752.

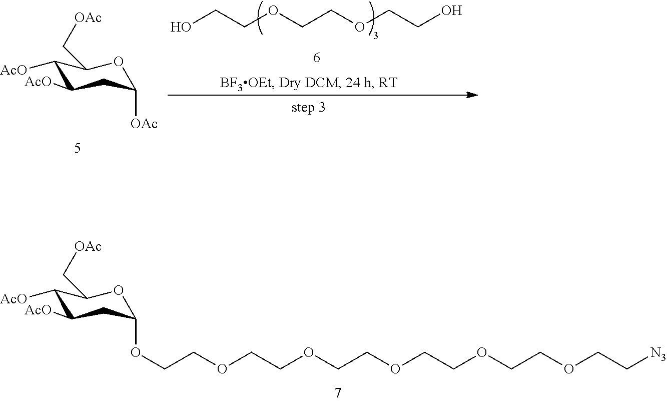

Synthesis of (2R,3S,4R,6S)-2-(acetoxymethyl)-6- ((17-azido-3,6,9,12,15-pentaoxaheptadecyl) oxy)tetrahydro-2H-pyran-3,4-diyl diacetate (7): Compound 5 (1.27 g, 1.0 eq, 3.82 mmol) was dried on high vacuum for 5 minutes, dissolved in 10 mL anhydrous DCM and the solution was stirred at 0° C. To this stirred solution was added boron trifluoride etherate (1.18 mL, 2.5 eq, 9.60 mmol) dropwise at 0° C. followed by drop wise addition of Azido-PEG6-Alcohol 6 (1.27 g, 1.0 eq, 3.82 mmol). The reaction mixture was allowed to stir at room temperature for 24h. The reaction was constantly monitored with the help of thin-layer chromatography (TLC) stained with H₂SO₄ dip. After maximum conversion on TLC, the reaction was quenched with ice. The reaction mixture was diluted with DCM and the organic layer was washed with saturated sodium bicarbonate solution (2×50 ml) and brine (2×50 ml). The combined organic layer was dried over Na₂SO₄, filtered, and evaporated in vacuo. The crude product was purified by silica flash column chromatography [ethyl acetate/hexane, 90:10 (v/v)] to afford compound 7 in 50% yield.

Viscous liquid; ¹H NMR (500 MHz, CDCl₃) δ 1.82 (td, J=12.47, 3.47 Hz, 1H), 1.98-2.11 (m, 9H), 2.26 (dd, J=13.04, 5.38 Hz, 1H), 3.39 (t, J=5.23 Hz, 2H), 3.58-3.77 (m, 22H), 4.00-4.05 (m, 2H), 4.31 (dd, J=12.56, 4.35 Hz, 1H), 4.98-5.02 (m, 2H), 5.30-5.36 (m, 1H). ¹³C NMR (125 MHz, CDCl₃) δ 20.77, 20.80, 21.01, 34.94, 50.68, 62.31, 66.84, 67.74, 69.08, 69.36, 70.04, 70.16, 70.58, 70.60, 70.63, 70.66, 70.68, 70.70, 77.23, 97.10, 169.96, 170.23, 170.81. (MALDI-TOF) m/z: calculated for C₂₄H₄₁N₃O₁₃ [M+Na]⁺602.2537 found 602.2526. Specific rotation: (α_{D}²⁰=53.8° (c=1, CHCl₃).

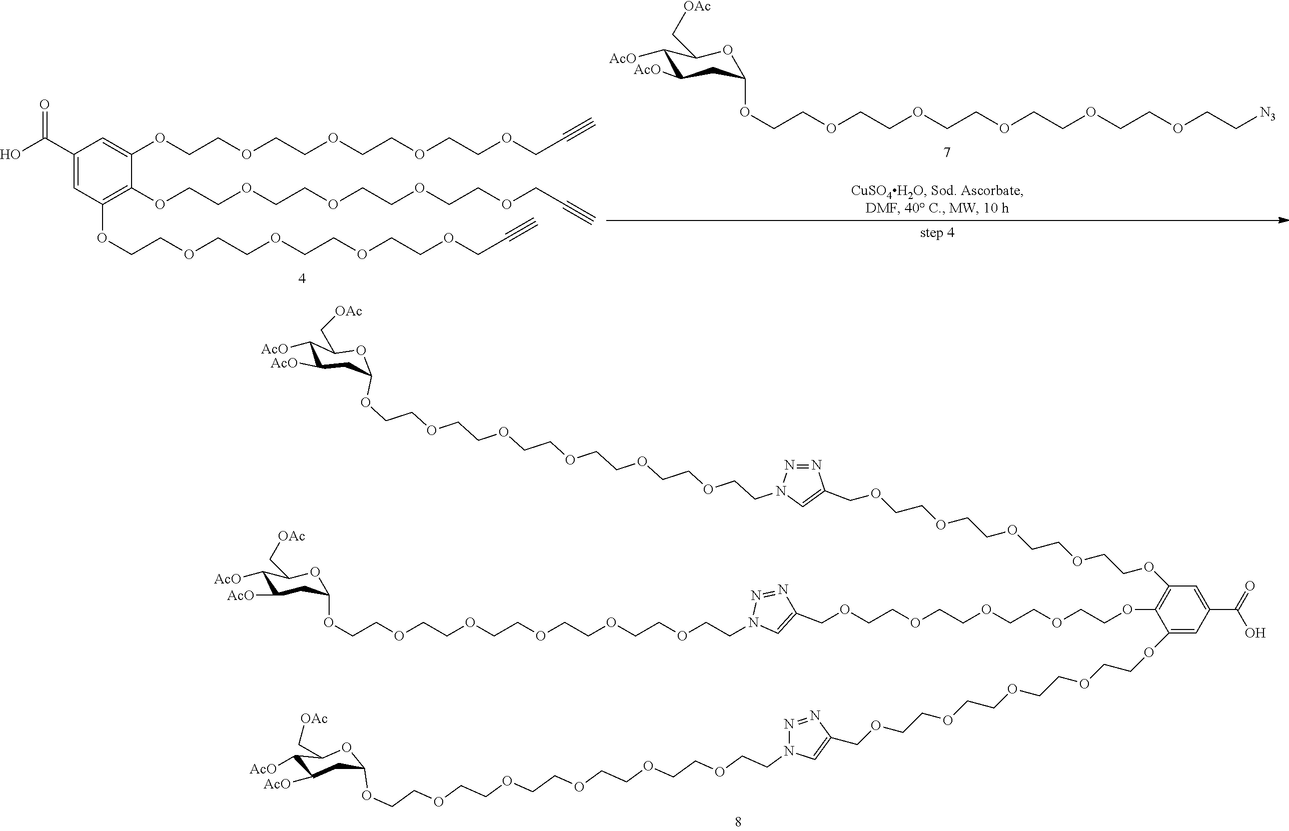

Synthesis of 3,4,5-tris((1-(1-(17- (((2S,4R,5S,6R)-4,5-diacetoxy-6-(acetoxymethyl)tetrahydro-2H-pyran-2-yl)oxy)-3,6,9,12,15-pentaoxaheptadecyl)-1H-1,2,3-triazol-4-yl)-2,5,8,11-tetraoxatridecan-13-yl)oxy)benzoic acid (8): To a stirred solution of compound 4 (280 mg, 1.0 eq, 0.344 mmol) in 4 mL DMF in a microwave vial was added solution of compound 7 (678 mg, 3.5 eq, 1.171 mmol) dissolved in 4 mL DMF followed by the addition of Cu_{S}O₄·5H₂O (5 mol % per acetylene) dissolved in 1 mL DI water. After 2 minutes, Sodium Ascorbate (10 mol % per acetylene) was added, and the reaction was performed under microwave irradiation at 40° C. for 10 h. Progress of the reaction was monitored with thin-layer chromatography (TLC). After complete conversion, the reaction mixture was diluted with DCM (100 mL) and washed with saturated EDTA solution (7×30 ml) and brine (1×50 ml), then dried (Na₂SO₄), filtered, and evaporated in vacuo. The crude product was purified by silica flash column chromatography [methanol/dichloromethane, 8:92 (v/v)] to afford compound 8 in 90% yield.

Viscous Liquid; ¹H NMR (500 MHz, CDCl₃) δ 1.80 (td, J=12.1, 3.6 Hz, 3H), 1.96-2.03 (m, 18H), 2.06 (s, 9H), 2.24 (dd, J=13.0, 5.4 Hz, 3H), 3.56-3.66 (m, 97H), 3.68-3.87 (m, 17H), 3.97-4.05 (m, 5H), 4.14-4.24 (m, 7H), 4.29 (dd, J=12.5, 4.3 Hz, 3H), 4.48-4.54 (m, 6H), 4.66 (d, J=12.6 Hz, 6H), 4.94-5.01 (m, 6H), 5.30 (ddd, J=12.2, 9.5, 5.2 Hz, 3H), 7.36 (s, 2H), 7.69-7.75 (m, 3H). ¹³C NMR (125 MHz, CDCl₃) δ 19.46, 19.76, 19.80, 19.82, 19.93, 20.00, 28.69, 28.94, 33.91, 35.10, 52.46, 61.30, 61.43, 61.51, 62.57, 63.06, 65.81, 66.05, 66.73, 67.70, 67.91, 68.06, 68.10, 68.33, 68.78, 69.14, 69.31, 69.44, 69.47, 69.52, 69.56, 69.61, 69.64, 69.73, 69.87, 70.93, 71.32, 76.26, 96.07, 97.60, 98.72, 108.61, 141.48, 151.18, 166.39, 168.94, 169.24, 169.81. (MALDI-TOF) m/z: calculated for C₁₁₂H₁₈₃N₉O₅₆ 2550.17 found 2550.18.

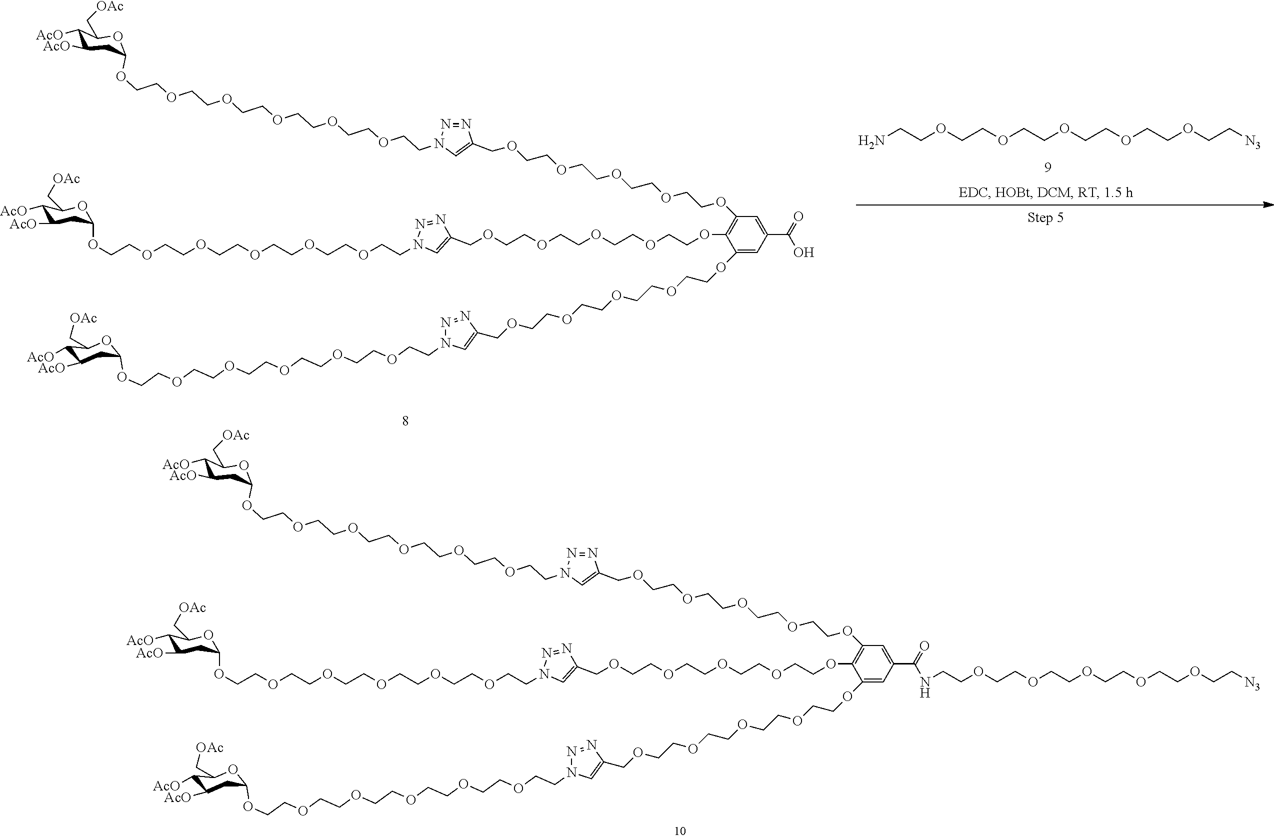

Synthesis of compound 10: Compound 8 (200 mg, 1.0 eq, 0.078 mmol) was dissolved in 4 mL Anhy. DCM and stirred at 0° C. EDC.HCl (22.54 mg, 1.5 eq, 0.1176 mmol) and HOBT (16.72 mg, 1.4 eq, 0.109 mmol) were added to the reaction mixture and stirred for 40 minutes. Compound 9 (47.55 mg, 2.0 eq, 0.157 mmol) was dissolved in 1 mL Anhy. DCM and was added to the reaction mixture. The Reaction mixture was allowed to stir at room temperature for 1.5 hours. Progress of reaction was monitored with the help of thin-layer chromatography (TLC) in UV light. The reaction mixture was diluted with DCM and the organic layer was washed with water (5×30 ml) and brine (2×30 ml), then dried (Na₂SO₄), filtered, and evaporated in vacuo. The crude product was purified by silica flash column chromatography [methanol/dichloromethane, 10:90 (v/v)] to afford compound 10 in 90% yield.

¹H NMR (500 MHz, DMSO-d₆) δ 1.72-1.83 (m, 3H), 1.93-2.05 (m, 27H), 2.06-2.14 (m, 3H), 3.35-3.43 (m, 5H), 3.43-3.74 (m, 127H), 3.72-3.89 (m, 7H), 3.90-4.08 (m, 5H), 4.08-4.22 (m, 7H), 4.43-4.63 (m, 11H), 4.80-4.90 (m, 2H), 4.96-5.02 (m, 2H), 5.07-5.20 (m, 2H), 7.19 (d, J=7.4 Hz, 2H), 7.97-8.12 (m, 3H), 8.46 (t, J=5.7 Hz, 1H, NH). ¹³C NMR (125 MHz, DMSO-d₆) δ20.90, 20.99, 21.11, 34.94, 49.75, 50.43, 55.40, 62.49, 63.97, 66.57, 67.70, 68.80, 68.96, 69.17, 69.36, 69.40, 69.50, 69.71, 69.82, 69.98, 70.07, 70.14, 70.21, 70.24, 70.26, 70.30, 70.41, 72.31, 96.60, 106.71, 106.72, 124.70, 144.24, 152.23, 165.99, 169.95, 170.21, 170.56.

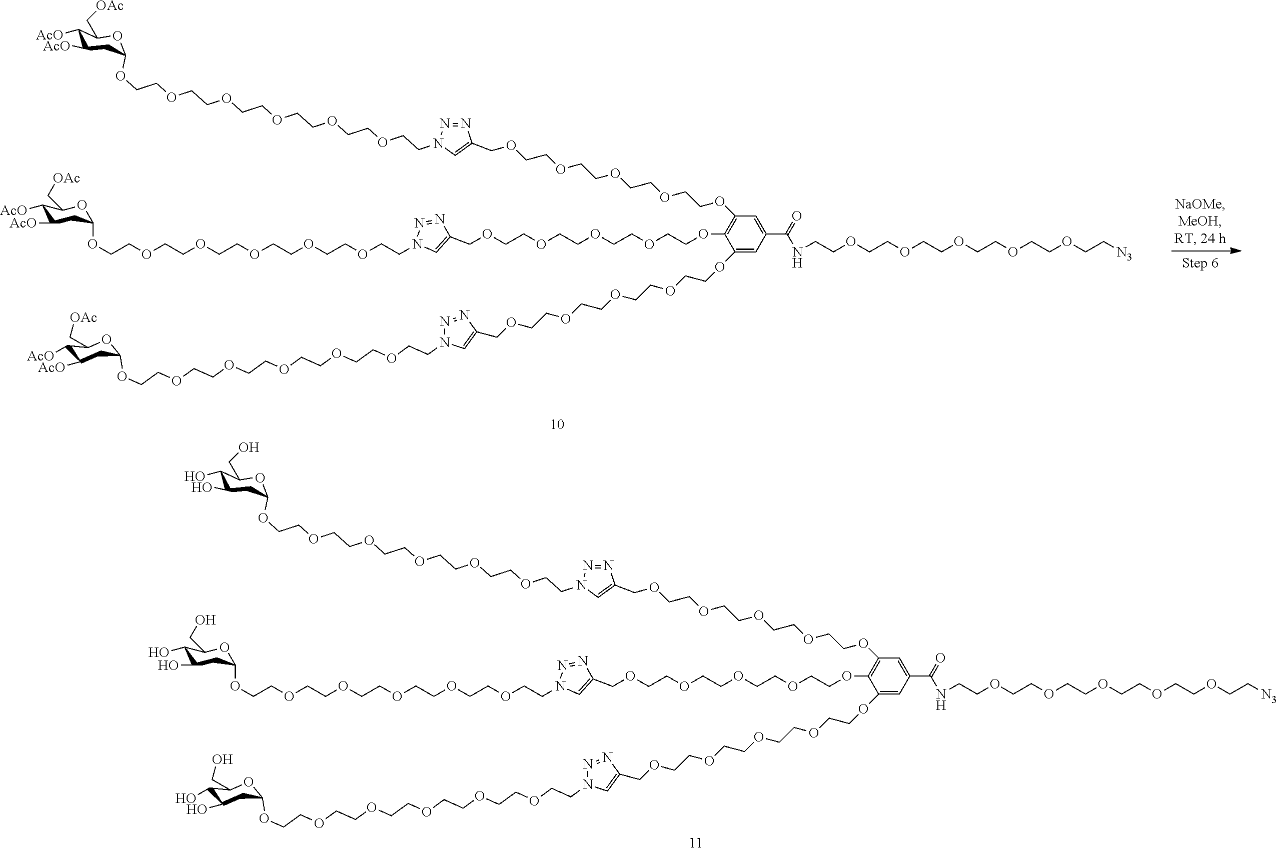

Synthesis of N-(17-azido-3,6,9,12,15-pentaoxaheptadecyl)-3,5-bis((1-(1-(17-(((2S,4R,5S,6R)-4,5-dihydroxy-6-(hydroxymethyl)tetrahydro-2H-pyran-2-yl)oxy)-3,6,9,12,15-pentaoxaheptadecyl)-1H-1,2,3-triazol-4-yl)-2,5,8,11-tetraoxatridecan-13-yl)oxy)-4-((1-(1-(17-(((2S,5S,6R)-4,5-dihydroxy-6-(hydroxymethyl)tetrahydro-2H-pyran-2-yl)oxy)-3,6,9,12,15-pentaoxaheptadecyl)-1H-1,2,3-triazol-4-yl)-2,5,8,11-tetraoxatridecan-13-yl)oxy)benzamide (11): To a stirred solution of compound 10 (1 g, 1.0 eq, 0.347 mmol) dissolved in dry MeOH (10 mL), sodium methoxide (300 mL, 1.1 M solution in MeOH) was added dropwise to adjust the pH around 8.5-9. The reaction mixture was stirred overnight at room temperature. The completion of the reaction was monitored by TLC. On completion, the pH was adjusted between 6 to 7 with an Amberlist IR-120 resin. The resin was removed by filtration, and the solvent was removed by rotary evaporation to yield compound 11 in 89%.

Viscous Liquid; ¹H NMR (500 MHz, DMSO-d₆) δ 1.39-1.47 (m, 3H), 1.84-1.93 (m, 3H), 2.98-3.06 (m, 3H), 3.17 (d, J=5.0 Hz, 2H), 3.33-3.70 (m, 133H), 3.75-3.82 (m, 9H), 4.04-4.16 (m, 6H), 4.42 (t, J=5.9 Hz, 3H), 4.45-4.59 (m, 12H), 4.73 (d, J=4.9 Hz, 3H), 4.82 (d, J=3.4 Hz, 3H), 4.85 (d, J=5.1 Hz, 3H), 7.19 (s, 2H), 8.05 (s, 3H), 8.46 (t, J=5.7 Hz, 1H). ¹H NMR (500 MHz, D₂O) δ 1.62 (dd, J=12.6, 3.8 Hz, 3H), 2.06 (dd, J=13.4, 5.3 Hz, 3H), 3.20-3.36 (m, 6H), 3.42-3.68 (m, 143H), 3.70-3.77 (m, 11H), 3.84 (dt, J=8.8, 4.5 Hz, 20H), 4.16 (p, J=4.4 Hz, 7H), 4.45-4.60 (m, 15H), 4.92 (d, J=3.5 Hz, 7H), 7.02-7.13 (m, 2H), 7.97 (d, J=7.6 Hz, 3H). ¹³C NMR (125 MHz, DMSO-d₆) δ 38.31, 40.35, 49.76, 50.43, 61.47, 63.94, 66.04, 67.82, 68.41, 68.80, 69.15, 69.37, 69.40, 69.47, 69.70, 69.98, 70.00, 70.06, 70.13, 70.20, 70.23, 70.25, 70.29, 70.40, 71.06, 72.05, 72.10, 72.30, 73.55, 77.51, 83.13, 97.12, 99.76, 106.70, 119.22, 124.72, 129.69, 140.34, 144.24, 152.23, 166.07. (MALDI-TOF) m/z: calculated for C₁₀₆H₁₈₉N₁₃O₅₁ [M−H]⁺: 2459.2517 found 2459.3218.

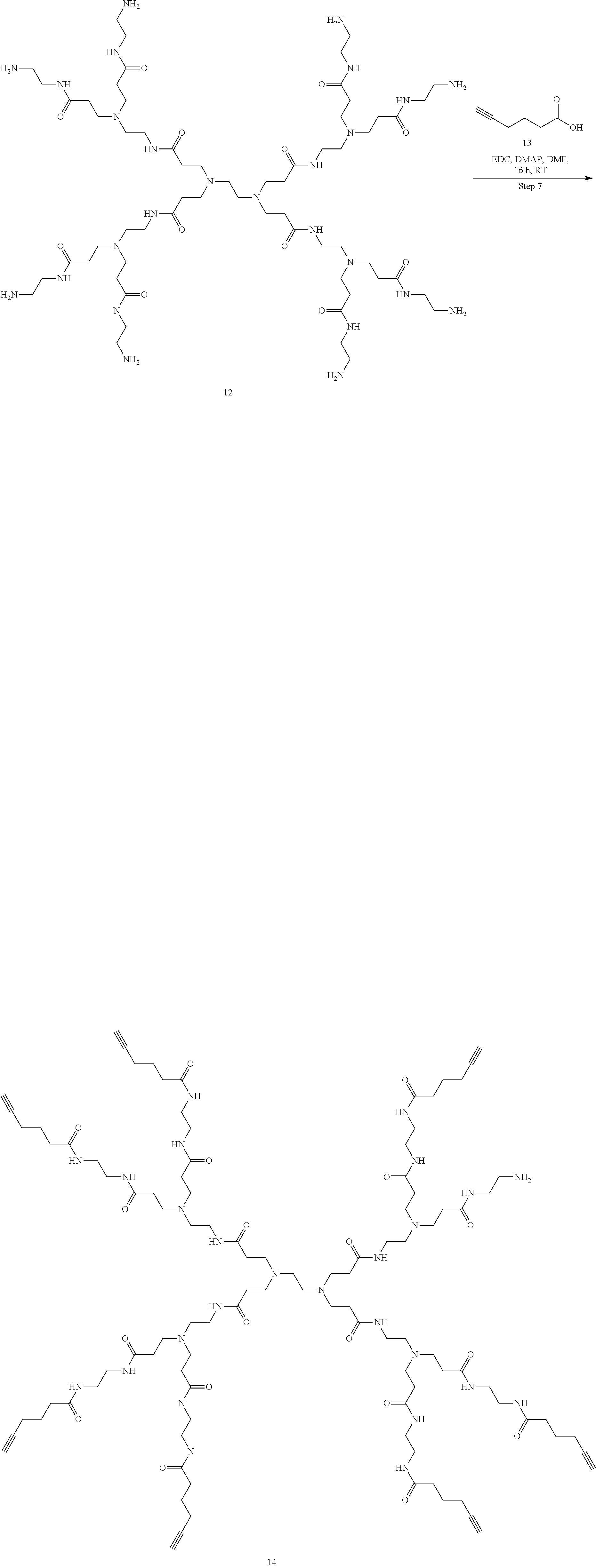

Synthesis of compound 14: Hexynoic Acid 13 (2 g, 24 eq, 18.48 mmol) was dissolved in Anhy. DMF (5 mL) and was activated by adding EDC (3.5 g, 24 eq, 18.48 mmol) and stirred for 15 minutes. It was then added dropwise to the G1 PAMAM dendrimer (1.1 g, 1.0 eq, 0.77 mmol) solution under continuous stirring followed by addition of DMAP (1.1g, 12 eq, 9.24 mmol) and the reaction mixture was stirred for 24 hours at room temperature. Upon completion, the dialysis was performed using a 1 kDa dialysis membrane in DMF for 12h. The final dialysis was performed against DI water. The aqueous solution was lyophilized to afford compound 14 in 84% yield.

¹H NMR (500 MHz, DMSO-d₆) δ 1.62-1.70 (m, 16H), 2.01-2.34 (m, 63H), 2.37-2.48 (m, 13H), 2.54-3.02 (m, 39H), 3.17 (s, 14H), 3.33 (s, 11H), 7.61-8.21 (m, 20H). ¹³C NMR (125 MHz, DMSO-d₆) δ 17.87, 24.65, 33.71, 34.64, 37.36, 38.78, 38.82, 50.01, 50.14, 52.61, 55.37, 71.90, 84.56, 171.73, 172.02, 172.13.

Synthesis of compound 15: To a stirred solution of compound 14 (52 mg, 1.0 eq, 0.024 mmol) in DMF (4 mL) in a microwave vial was added solution of compound 10 (613 mg, 9.0 eq, 0.191 mmol) dissolved in DMF (4 mL). To this solution, CuSO₄·5H₂O (6.0 mg, 1.0 eq, 0.024 mmol) and sodium ascorbate (7.0 mg, 1.5 eq, 0.037 mmol) dissolved in minimum amount of deionized water were added. The reaction was performed in microwave condition at 40° C. for 12 h. The completion of reaction was tracked using HPLC. Upon completion, the dialysis was performed using a 3.5 kDa dialysis membrane in DMF for 12 h. The final dialysis was performed in DI water. The product was lyophilized to afford compound 15 in 90% yield.

¹H NMR (500 MHz, DMSO-d₆) δ 1.72-1.78 (m, 24H), 1.93-2.01 (m, 216H), 2.10 (dd, J=13.0, 5.5 Hz, 24H), 3.34-3.62 (m, 1024H), 3.63-3.70 (m, 44H), 3.70-3.88 (m, 108H), 3.87-4.10 (m, 68H), 4.09-4.22 (m, 60H), 4.40-4.61 (m, 116H), 4.84 (t, J=9.8 Hz, 26H), 4.99 (d, J=3.3 Hz, 22H), 5.06-5.19 (m, 24H), 7.18 (s, 16H), 7.82 (s, 7H, NH), 8.04 (s, 24H, triazole-H), 8.42-8.50 (m, 8H, triazole-H). ¹³C NMR (125 MHz, DMSO-d₆) δ 20.89, 20.98, 21.11, 34.93, 49.75, 62.49, 63.97, 66.57, 67.70, 68.81, 68.96, 69.16, 69.36, 69.40, 69.82, 69.98, 70.07, 70.13, 70.21, 70.24, 70.26, 70.30, 70.30, 70.41, 72.32, 96.60, 106.71, 124.74, 152.24, 169.96, 170.22, 170.57.

Synthesis of compound 16 (2DG-D): To a stirred solution of compound 14 (483 mg, 1.0 eq, 0.222 mmol) in DMF (5 mL) in a microwave vial was added solution of compound 11 (5 mg, 9.0 eq, 2 mmol) dissolved in deionized water (5 mL). To this solution, CuSO₄·5H₂O (55.0 mg, 1.0 eq, 0.222 mmol) and sodium ascorbate (66.6 mg, 1.5 eq, 0.333 mmol) dissolved in minimum amount of deionized water was added. The reaction vial was irradiated under microwave conditions at 80° C. for 15 h. The completion of reaction was tracked using HPLC. Upon completion, the dialysis was performed using a 3.5 kDa dialysis membrane in DMF for 12h. The final dialysis was performed in D.I. water. The product was lyophilized to afford compound 16 in 95% yield.

¹H NMR (500 MHz, DMSO-d₆) δ 1.44 (dd, J=12.3, 3.6 Hz, 24H), 1.87 (dd, J=12.9, 5.2 Hz, 24H), 2.97-3.13 (m, 60H), 3.47-3.70 (m, 1175H), 3.71-3.90 (m, 155H), 4.04-4.20 (m, 53H), 4.38-4.56 (m, 132H), 4.70-4.93 (m, 65H), 7.18 (s, 16H), 7.78-7.91 (m, 12H), 8.04 (s, 24H), 8.47 (t, J=5.7 Hz, 8H). ¹³C NMR (125 MHz, D₂O) δ 36.58, 37.19, 39.42, 39.55, 49.94, 60.29, 60.59, 60.88, 63.02, 65.99, 67.90, 68.12, 68.34, 68.66, 68.83, 68.91, 69.10, 69.39, 69.41, 69.49, 69.53, 69.55, 69.58, 69.61, 69.66, 69.83, 70.12, 70.40, 70.77, 70.92, 71.08, 71.67, 71.94, 72.19, 75.95, 91.23, 93.33, 97.32, 106.28, 125.42, 139.68, 151.88.

Synthesis of compound 17: To a stirred solution of compound 4 (200 mg, 1.0 eq, 0.246 mmol) in 4mL dry DCM was added EDC.HCl (70 mg, 1.5 eq, 0.369 mmol) and HOBt (46 mg, 1.4 eq, 0.344 mmol) at 0° C. and was stirred for 40 minutes. The compound 9 (150.7 mg, 2.0 eq, 0.492 mmol) was dissolved in 1 mL dry DCM and was added to the reaction mixture. The Reaction mixture was allowed to stir at room temperature for 1.5 hours. Progress of reaction was monitored with the help of thin-layer chromatography (TLC) in UV light. The reaction mixture was diluted with DCM and the organic layer was washed with water (5×30 ml) and brine (2×30 ml), then dried (Na₂SO₄), filtered, and evaporated in vacuo. The crude product was purified by silica flash column chromatography [methanol/dichloromethane, 7:93 (v/v)] to afford compound 17 in 83% yield. ¹H NMR (500 MHz, DMSO-d₆) δ 8.47 (t, 1H, J=5.7 Hz, —NH), 7.18 (s, 2H, Ar—H), 4.19-4.04 (m, 11H, PEG-H), 3.81-3.73 (m, 4H, PEG-H), 3.67 (t, 2H, J=4.9 Hz, PEG-H), 3.65-3.46 (m, 44H, PEG-H), 3.43-3.36 (m, 9H, PEG-H), 3.21-3.13 (m, 2H, PEG-H). ¹H NMR (500 MHz, CDCl₃) δ 7.03 (s, 2H, Ar—H), 4.19-4.08 (m, 13H, PEG-H), 3.84-3.53 (m, 72H, PEG-H), 3.29 (t, J=5.0 Hz, 2H, PEG-H), 2.38 (t, J=2.4 Hz, 3H, —CH). ¹³C NMR (125 MHz, DMSO-d₆) δ 166.1, 152.2, 140.3, 129.7, 106.7, 80.8, 77.5, 72.3, 70.4, 70.31, 70.3, 70.3, 70.25, 70.23, 70.2, 70.1, 69.9, 69.7, 69.4, 69.3, 69.0, 68.8, 57.9, 50.4, 49.1.

Synthesis of compound 18: The compound 17 (200 mg, 0.182 mmol, 1.0 eq) was dissolved in dry THF (6 mL) and cooled to −78° C. n-BuLi (1.6M in Hexanes, 0.56mL, 0.9 mmol, 5.0eq) was added dropwise to the solution, and the mixture was stirred for 30 minutes at −78° C. followed by the dropwise addition of Triisopropylsilyl chloride (TIPS-Cl) (351.5 mg, 1.82 mmol, 10.0 eq) at −78° C. The Reaction mixture was allowed to come to room temperature and stirred for 5 hours. Progress of the reaction was monitored with the help of thin-layer chromatography (TLC) using UV light. The reaction mixture was quenched with saturated ammonium chloride solution, diluted with DCM and the organic layer was washed with water (3×30 ml) and brine (2×30 ml), then dried (Na₂SO₄), filtered, and evaporated in vacuo. The crude product was purified by silica flash column chromatography [methanol/dichloromethane, 5:95 (v/v)] to afford compound 18 in 71% yield.

¹H NMR (500 MHz, CDCl₃) δ 7.02 (s, 2H, Ar—H), 4.16 (s, 6H, PEG-H), 4.15-4.10 (m, 6H, PEG-H), 3.80-3.75 (m, 6H, PEG-H), 3.74-3.69 (m, 5H, PEG-H), 3.68-3.50 (m, 70H, PEG-H), 1.19 (s, 9H, 9×—CH), 1.00 (d, 66H, 18×—CH₃). ¹³C NMR (125 MHz, CDCl₃) δ 166.0, 151.4, 106.1, 102.2, 86.6, 71.3, 70.3, 69.7, 69.6, 69.6, 69.52, 69.5, 69.3, 69.2, 69.0, 68.9, 68.6, 67.9, 67.6, 58.10, 49.6, 41.7, 38.9, 28.7, 17.5, 16.9, 10.9, 10.3, 10.1, 9.9.

Synthesis of compound 19: To a stirred solution of compound 18 (120 mg, 0.076 mmol, 9.0 eq) in 1 mL dry DMF in a microwave vial was added solution of compound 14 (18.5 mg, 0.0085 mmol, 1.0 eq) dissolved in 1 mL dry DMF followed by the addition of copper bromide (5 mol % per acetylene) and N,N,N′,N″,N″-Pentamethyldiethylenetriamine (PMDETA) (10 mol % per acetylene) was added, and the reaction vial was irradiated under microwave at 40° C. for 10h. Upon completion, the dialysis was performed using a 2 kDa dialysis membrane against D.I. water for 12 h. The aqueous solution was lyophilized to afford final compound 19 in 75% yield.

¹H NMR (500 MHz, DMSO-d₆) δ 8.47 (t, 8H, J=5.8 Hz, 8-amide-H), 8.08-7.62 (m, 30H, Triazole-H+amide-H), 7.19 (s, 16H, Ar—H), 5.41 (s, PEG-H), 4.45 (s, PEG-H), 4.26-3.93 (m, PEG-H), 3.88-3.36 (m, PEG-H), 3.18-2.97 (m, PEG-H), 2.75-2.55 (m, core-CH₂), 2.34-1.96 (m, core-CH₂), 1.87-1.57 (m, core-CH₂), 1.52-1.07 (m, TIPS-H), 1.10 (m, TIPS-H). ¹³C NMR (125 MHz, DMSO) δ 166.0, 152.2, 140.3, 106.7, 104.9, 86.6, 79.6, 79.4, 79.1, 72.3, 71.0, 70.4, 70.3, 70.25, 70.2, 70.1, 69.9, 69.5, 69.4, 68.8, 68.7, 58.6, 44.0, 40.4, 18.8, 18.2, 11.8, 11.2, 11.0.

Synthesis of compound 20: To a stirred solution of compound 19 (200 mg, 0.014 mmol, 1.0 eq) dissolved in THF (5 mL), 0.67 mL tetra-n-butylammonium fluoride (TBAF) (1M in THF, 0.67 mmol, 48.0 eq) was added dropwise. The reaction mixture was stirred overnight at room temperature. Upon completion, the dialysis was performed using a 1 kDa dialysis membrane against 50:50 D.I. water: DMF for 12 h. The final dialysis was performed against D.I. water. The aqueous solution was lyophilized to afford final compound 20 in 79% yield.

¹H NMR (500 MHz, DMSO-d₆) δ 8.14 (s, 8H, 8-amide-H), 7.31 (s, 16H, Ar—H), 4.75-4.35 (m, PEG-H), 4.34-4.14 (m, PEG-H), 4.07-3.76 (m, PEG-H), 3.76-3.66 (m, PEG-H), 3.61-3.46 (m, PEG-H), 2.90-2.69 (m, core-CH₂+alkyne), 2.33-2.22 (m, core-CH₂), 2.00-1.89 (m, core-CH₂). ¹³C NMR (125 MHz, DMSO-d₆) δ 172.5, 166.0, 152.2, 140.3, 129.7, 106.7, 73.5, 72.8, 72.3, 70.4, 70.3, 70.3, 70.22, 70.2, 70.1, 70.07, 70.0, 69.5, 69.4, 69.2, 68.8, 60.6, 49.9, 38.5, 35.3, 25.5.

Synthesis of compound 21: To a stirred solution of compound 7 (1g, 1.72 mmol, 1.0 eq) dissolved in dry MeOH (10 mL), sodium methoxide (0.3 mL, 1.1 M solution in MeOH) was added dropwise to adjust the pH around 8.5-9. The reaction mixture was stirred overnight at room temperature. The completion of the reaction was monitored by TLC. On completion, the pH was adjusted between 6 to7 with an Amberlist IR-120 resin. The resin was removed by filtration, and the solvent was removed by rotary evaporation to yield compound 21 in 86%.

¹H NMR (500 MHz, D₂O) δ 4.92 (d, J=3.4 Hz, 1H, Sugar-H), 3.84-3.77 (m, 1H, Sugar-H), 3.76-3.70 (m, 2H, Sugar-H), 3.68-3.64 (m, 1H, Sugar-H), 3.63-3.58 (m, 22H, PEG-H), 3.55-3.50 (m, 2H, Sugar-H), 3.41-3.37 (m, 2H, PEG-H), 3.29-3.22 (m, 1H, Sugar-H), 2.06 (ddd, J=13.4, 5.2, 1.3 Hz, 1H, Sugar-H), 1.64-1.55 (m, 1H, Sugar-H). ¹³C NMR (125 MHz, D₂O) δ 97.3, 72.2, 71.0, 69.5, 69.47, 69.2, 68.1, 66.0, 60.5, 50.1, 36.5.

Synthesis of compound 16 (Divergent Route): To a stirred solution of compound 20 (100 mg, 0.09 mmol, 1.0 eq) in DMF (4 mL) in a microwave vial was added solution of compound 21 (123.8 mg, 0.27 mmol, 30.0eq) dissolved in DI Water (4 mL). To this solution, CuSO₄·5H₂O (5 mol % per acetylene) dissolved in 0.1 mL DI water was added. After 2 minutes, Sodium Ascorbate (10 mol % per acetylene) was added, and the reaction vial was heated under microwave irradiation at 50° C. for 12 h. Upon completion, the dialysis was performed using a 1 kDa dialysis membrane against pure water for 12 h. The aqueous solution was lyophilized to afford compound 16 in 83% yield.

¹H NMR (500 MHz, DMSO) δ 8.47 (t, 8H J=5, 6 Hz, 8-triazole-H), 8.04 (m, 52H, Triazole-H+amide-H), 7.18 (s, 16H, Ar—H), 4.95-4.69 (m, Sugar-H), 4.60-4.34 (m, Sugar-H), 4.21-4.02 (m, Sugar-H+PEG-H), 3.92-3.74 (m, PEG-H), 3.70-3.46 (m, PEG-H), 3.17-2.89 (m, PEG-H+Core-CH₂), 2.67-2.51 (m, Core-CH₂), 2.11 (s, 16H, Core-CH₂), 2.02-1.63 (m, 40H, Sugar-H+Core-CH₂), 1.44 (m, 24H, Sugar-H).

### Trehalose Dendrimer

Synthesis of methyl 3,4,5-tris((3,6,9,12-tetraoxapentadec-14-yn-1-yl)oxy)benzoate (3) (New Route): Compound 22 (2g, 1.0 eq, 2.81 mmol) was dissolved in anhydrous DMF (20 mL). To the stirring solution, sodium hydride (505 mg, 4.5 eq, 12.64 mmol, 60% dispersion in mineral oil) was slowly added in portions at 0° C. The solution was stirred for 15 min at 0° C. This was followed by the addition of propargyl bromide (1.06 mL, 4.0 eq, 11.22 mmol, 80% w/w solution in toluene) at 0° C., and the stirring continued at room temperature for another 3 h. On completion, the reaction was quenched by the addition of saturated ammonium chloride solution and extracted with ethyl acetate (2×150 ml). The combined organic layer was washed with chilled brine (2×100 ml), dried over Na₂SO₄, and evaporated in vacuo. The crude product was purified by silica flash column chromatography to afford compound 3 in 75% yield (1.7 g) as a brown viscous liquid.

¹H NMR (500 MHz, CDCl₃) δ 7.29 (s, 2H), 4.17-4.25 (m, 12H), 3.84-3.90 (m, 7H), 3.78 (t, J=5.2 Hz, 3H), 3.63-3.72 (m, 35H), and 2.43 (s, 3H). ¹³C NMR (125 MHz, DMSO-d₆) δ 166.3, 152.5, 142.3, 124.8, 108.5, 80.8, 77.6, 77.5, 72.8, 72.4, 70.5, 70.4, 70.3, 70.2, 70.1, 70.0, 69.4, 69.0, 68.9, 60.6, 57.94, 52.6. (MALDI-TOF) m/z: calculated for C₄₁H₆₂O₁₇ [M+Na]⁺: 849.3885; found 849.3870.

Synthesis of (2R,3R,4S,5R,6R)-2-(acetoxymethyl)-6-(((2R,3R,4S,5R,6S)-3,4,5-triacetoxy-6-(azidomethyl)tetrahydro-2H-pyran-2-yl)oxy)tetrahydro-2H-pyran-3,4,5-triyl triacetate (23): The compound (2R,3R,4S,5R,6R)-2-(acetoxymethyl)-6-(((2R,3R,4S,5S,6R)-3,4,5-triacetoxy-6-(bromomethyl)tetrahydro-2H-pyran-2-yl)oxy)tetrahydro-2H-pyran-3,4,5-triyl triacetate (18.66 g, 26.63 mmol) was dissolved in dry DMF (280 mL) and stirred under a nitrogen environment. Solid sodium azide (8.69 g, 133.63 mmol) was added to the reaction mixture and the solution was heated at 55° C. and stirred for 16 h. At completion of reaction, the solvent was removed under reduced pressure and the crude was dissolved in EtOAc (500 mL). The organic layer was washed 3×150 mL with water and (3×100 mL) brine. The organic layer was dried with anhydrous Na₂SO₄ and concentrated under reduced pressure. The crude brown-solid was purified by column chromatography on silica gel to obtain 12.20 g of pure 23 as a white solid with 69.1% yield (12.2g) as a white solid.

¹H NMR (500 MHz, DMSO-d₆) δ 5.20-5.38 (m, 4H), 4.97-5.09 (m, 4H), 4.16-4.23 (m, 1H), 3.94-4.06 (m, 3H), 3.47-3.54 (m, 1H), 3.39-3.46 (m, 1H), and 1.98-2.10 (m, 21H). ¹³C NMR (125 MHz, DMSO-d₆) δ 170.5, 170.2, 170.0, 169.8, 92.5, 92.4, 79.6, 70.0, 69.6, 69.5, 69.1, 68.5, 68.4, 62.2, 50.3, 40.4, 40.2, 40.1, 20.9, 20.9, 20.8, 20.8.

Synthesis of compound 24: To a stirred solution of compound 4 (614 mg, 1.0 eq, 0.75 mmol) in 5 mL of DMF in a microwave vial was added the solution of compound 23 (1.7 g, 3.5 eq, 2.57 mmol) dissolved in 5 mL of DMF. It was followed by the addition of CuSO₄·5H₂O (5 mol % per acetylene) dissolved in 1 mL of deionized water. After stirring for 2 minutes, sodium ascorbate (10 mol % per acetylene) solution in 0.5 mL of deionized water was added to the reaction vial and was irradiated in a microwave at 50° C. for 10 h. Progress of the reaction was monitored with TLC. After complete conversion, the reaction mixture was diluted with DCM (100 mL) and washed with saturated EDTA solution (7×30 ml) and brine (1×50 ml), then dried (Na₂SO₄), filtered, and evaporated in vacuo. The crude product was purified by silica flash column chromatography to afford compound 24 as white solid in 90% yield (1.8 g).

¹H NMR (500 MHz, DMSO-d₆) δ 8.00 (s, 3H), 7.21 (s, 2H), 5.28 (m, 10H), 5.01 (m, 14H), 4.84 (m, 3H), 4.65 (d, J=14.4 Hz, 3H), 4.50 (m, 10H), 4.17 (m, 9H), 4.10 (m, 6H), 3.98 (m, 8H), 3.75 (m, 10H), 3.66 (t, J=4.8 Hz, 2H), 3.60 (m, 4H), 3.53 (m, 29H), 2.05 (m, 20H), and 1.98 (m, 38H). ¹³C NMR (125 MHz, CDCl₃) δ 170.7, 170.6, 170.0, 169.9, 169.8, 169.7, 169.6, 169.5, 169.4, 162.6, 145.5, 92.2, 91.5, 72.3, 70.9, 70.8, 70.7, 70.6, 70.5, 70.4, 70.0, 69.9, 69.8, 69.7, 68.9, 68.5, 68.3, 61.7, 36.5, 31.4, 20.9, 20.8, 20.7, 20.6, 20.5. (MALDI-TOF) m/z: calculated for C₁₂₀H₁₆₇N₉O₇₀ [M−H]⁺2795.97 found 2794.97.

Synthesis of compound 25: Compound 24 (200 mg, 1.0 eq, 0.08 mmol) was dissolved in 4 mL of anhydrous DCM and stirred at 0° C. EDC.HCl (22.54 mg, 1.5 eq, 0.12 mmol) and HOBT (16.72 mg, 1.4 eq, 0.11 mmol) were added to the reaction mixture and the reaction mixture was stirred for 30 minutes. To this stirring solution, compound 9 (47.55 mg, 2.0 eq, 0.16 mmol) in 1 mL of dry DCM was added. The reaction mixture was allowed to stir at room temperature for 1.5 h. Progress of the reaction was monitored with the help of TLC using UV light. At the completion of reaction, the reaction mixture was diluted with DCM and the organic layer was washed with water (5×30 ml) and brine (2×30 ml), then dried using anhydrous Na₂SO₄, followed by filtration and evaporation under reduced pressure. The crude product was purified by silica flash column chromatography to afford compound 24 (178 mg) as white solid in 85% yield.

¹H NMR (500 MHz, DMSO-d₆) δ 8.46 (t, 1H), 8.00 (s, 3H), 7.18 (s, 2H), 5.27 (m, 10H), 5.00 (m, 13H), 4.84 (d, J=3.6 Hz, 3H), 4.65 (dd, J=14.5, 2.9 Hz, 4H), 4.50 (d, J=16.9 Hz, 10H), 4.16 (m, 12H), 3.98 (m, 10H), 3.75 (m, 6H), 3.66 (t, J=5.0 Hz, 2H), 3.51 (m, 53H), and 1.97 (m, 63H). ¹³C NMR (125 MHz, CDCl₃) 175.2, 175.0, 174.9, 174.7, 174.6, 174.5, 170.7, 156.9, 149.2, 145.1, 134.5, 130.2, 111.5, 96.5, 96.2, 77.0, 75.2, 75.1, 75.0, 74.9, 74.8, 74.7, 74.6, 74.4, 74.3, 74.2, 74.1, 74.0, 73.8, 73.5, 73.4, 73.2, 73.1, 68.5, 66.9, 55.2, 54.8, 25.7, 25.6, 25.5, 25.5, 25.4.

Synthesis of compound 26: To a stirred solution of compound 25 (1 g, 1.0 eq, 0.32 mmol) dissolved in dry MeOH (10 mL), a dropwise addition of sodium methoxide (300 μL, 1.1 M solution in MeOH) was done to adjust the pH approximately 8.5-9. The reaction mixture was stirred overnight at room temperature. The completion of the reaction was monitored by TLC. On completion, the pH was adjusted between 6 to7 with an Amberlite IR-120 resin. The resin was removed by filtration, and the solvent was removed by rotary evaporation to yield compound 26 in 92% yield (655 mg).

¹H NMR (500 MHz, D₂O) δ 8.05 (m, 3H), 7.16 (m, 2H), 5.09 (m, 3H), 4.65 (m, 12H), 4.26 (m, 4H), 4.11 (m, 4H), 3.93 (m, 4H), 3.74 (m, 90H), and 3.35 (m, 6H). ¹³C NMR (125 MHz, D₂O) δ 169.3, 151.9, 143.7, 139.6, 126.0, 106.3, 93.3, 93.1, 72.5, 72.3, 72.1, 70.9, 70.8, 70.2, 70.1, 69.9, 69.8, 69.7, 69.6, 69.5, 69.4, 69.1, 68.8, 68.3, 62.9, 60.4, 50.7, 50.0, 39.6.

Synthesis of compound 27: To a stirred solution of compound 14 (7.0 mg, 1.0 eq, 0.0032 mmol) in DMF (1 mL) in a microwave vial was added a solution of compound 25 (100 mg, 10.0 eq, 0.033 mmol) dissolved in DMF (2 mL). To this solution, CuSO₄·5H₂O (5 mol % per acetylene) and sodium ascorbate (10 mol % per acetylene) dissolved in minimum amount of deionized water were added. The reaction vial was irradiated using microwave heating at 40° C. for 12 h. Formation of desired product was observed via HPLC analysis. Upon completion, the reaction mixture was transferred to a 3.5 kDa dialysis membrane and the dialysis was performed in DMF for 12 h. The final dialysis was performed in deionized water to exchange DMF. The aqueous solution was lyophilized to afford acetylated-trehalose (27) dendrimer in 90% yield.

¹H NMR (500 MHz, DMSO-d₆) δ 8.41 (t, J=5.8 Hz, Tre-D-amides), 7.94 (m, Tre-D-amides+triazoles), 7.85 (m, 3H), 7.75 (s, 4H), 7.11 (s, 16H, Tre-D-Ar—H), 5.21 (m, sugar-H), 4.94 (m, sugar-H), 4.78 (d, J=4.0 Hz, sugar-H), 4.58 (dd, J=14.5, 2.9 Hz, sugar-H), 4.41 (m, sugar-H), 4.10 (m, sugar-H), 3.96 (m, sugar-H+PEG-H), 3.87 (m, sugar-H+PEG-H), 3.69 (m, sugar-H+core+PEG), 3.59 (t, J=5.0 Hz, sugar-H+core+PEG), 3.51 (m, sugar-H+core), 3.43 (m, PEG-H), 3.00 (s, core-H), 1.91 (m, acetates), 1.17 (s, sugar-H). ¹³C NMR (125 MHz, DMSO-d₆) δ 170.6, 170.2, 170.0, 152.2, 124.7, 106.7, 96.6, 72.6, 70.5, 70.4, 70.3, 70.2, 70.13, 70.1, 70.0, 69.8, 69.4, 69.3, 69.1, 68.9, 68.8, 67.7, 66.6, 63.9, 62.5, 49.7, 34.9, 21.1, 21.0, 20.9

### Synthesis of Compound 28

Route 1: To a stirred solution of acetylated-trehalose dendrimer 27 (1.0 g, 1.0 eq, 0.32 mmol) in dry MeOH (10 mL), sodium methoxide (300 μL, 1.1 M solution in MeOH) was added dropwise to adjust the pH approximately 8.5-9. The reaction mixture was stirred overnight at room temperature. The completion of the reaction was monitored by TLC. On completion, the pH was adjusted between 6 and 7 with an Amberlite IR-120 resin. The resin was removed by filtration, and the solvent was removed by rotary evaporation to yield trehalose-dendrimer 28 in 95% yield. Route 2: To a stirred solution of compound 14 (26.6 mg, 1.0 eq, 0.012 mmol) in DMF (2 mL) in a microwave vial was added the solution of compound 26 (230 mg, 10.0 eq, 0.12 mmol) dissolved in deionized water (3 mL). To this solution, CuSO₄·5H₂O (5 mol % per acetylene) and sodium ascorbate (10 mol % per acetylene) dissolved in minimum amount of deionized water were added. The reaction vial was irradiated using microwave heating at 80° C. for 15 h. The formation of the product was observed with the help of HPLC. Upon completion, the dialysis was performed to purify the desired dendrimer using a 3.5 kDa dialysis membrane in DMF for 12 h. The final dialysis was performed in DI water. The aqueous solution was lyophilized to afford compound 28 in 86% yield (205 mg).

¹H NMR (500 MHz, DMSO-d₆) δ 8.48 (t, J=5.8 Hz, 8H, Tre-D-amides), 8.09-7.75 (m, 54H, Tre-D-core-amides+triazoles), 7.18 (s, 16H, D-Ar—H), 5.32 (s, sugar-H), 4.97 (s, sugar-H), 4.87-4.68 (m, sugar-H), 4.67-4.25 (m, sugar-H), 4.21-3.98 (m, PEG-H+sugar-H), 3.91-3.72 (m, PEG-H), 3.71-3.52 (m, PEG-H), 3.51-3.26 (m, PEG-H+core-H), 3.26-3.14 (m, core-H), 3.13-3.02 (m, core-H), 2.98 (t, J=9.3 Hz, Core-H), 2.82-2.52 (m, core-H), 2.34-2.02 (m, 42H, sugar-H), 1.84-1.75 (m, 16H, sugar-H).

¹H NMR (500 MHz, D₂O) δ 7.89 (m, Tre-d-triazoless), 7.06 (s, 16H, Tre-D-Ar), 5.02 (t, J=3.3 Hz, sugar-H), 4.55 (m, PEG-H), 3.58 (m, PEG-H), 2.35 (m, sugar-H), 1.80 (m, sugar-H), and 1.22 (s, sugar-H). ¹³C NMR (125 MHz, DMSO-d₆) δ 166.0, 152.2, 144.1, 140.3, 129.7, 124.9, 124.7, 122.4, 106.7, 93.7, 73.2, 73.1, 72.3, 71.9, 71.8, 70.5, 70.4, 70.3, 70.2, 70.1, 69.4, 69.2, 68.8, 63.8, 61.2, 51.2, 49.6, 49.1, and 29.5.

What is claimed is:

1. A dendrimer comprising:
(a) a central core; and
(b)(i) at least one 2-deoxyglucose-surfaced branching units conjugated to the central core; or
(b)(ii) at least one trehalose-surfaced branching units conjugated to the central core; or
(b)(iii) at least one 2-deoxyglucose-surfaced branching units conjugated to the central core and at least one trehalose-surfaced branching units conjugated to the central core.

2. The dendrimer of claim 1, wherein all the branching units present in the dendrimer are 2-deoxyglucose-surfaced branching units.

3. The dendrimer of claim 1, wherein all the branching units present in the dendrimer are trehalose-surfaced branching units.

4. The dendrimer of claim 1, wherein the central core comprises polyamidoamine, cyclodextrin, polylysine, 2,2-bismethylolpropionic, tetrazine, poly(propylene imine), polyethylene glycol, glycol, or adamantane.

5. The dendrimer of claim 1, wherein the branching units include at least one focal point selected from triazole, gallic acid, an amino acid, a peptide, a hydrocarbon, a linear polymer of an oligonucleotide.

6. The dendrimer of claim 1, wherein the branching units include repeating units selected from polyethylene glycol, a hydrocarbon, an amino acid, a peptide, a linear polymer, or an oligonucleotide.

7. The dendrimer of claim 1, wherein the dendrimer includes 1 to 500 2-deoxyglucose-surfaced branching units, or 1 to 500 trehalose-surfaced branching units.

8. A dendrimer comprising:
(a) a core;
(b) at least one interior layer comprising repeating units or mixed layers; and
(c)(i) 2-deoxyglucose terminal surface groups attached to an outermost interior layer; or
(c)(ii) trehalose terminal surface groups attached to an outermost interior layer; or
(c)(iii) 2-deoxyglucose terminal surface groups attached to an outermost interior layer and trehalose terminal surface groups attached to an outermost interior layer.

9. The dendrimer of claim 8, wherein the entire periphery of the dendrimer consists of 2-deoxyglucose terminal surface groups.

10. The dendrimer of claim 8, wherein the entire periphery of the dendrimer consists of trehalose terminal surface groups.

11. The dendrimer of claim 8, wherein the core comprises polyamidoamine, cyclodextrin, polylysine, 2,2-bismethylolpropionic, tetrazine, poly(propylene imine), polyethylene glycol, glycol, or adamantane.

12. The dendrimer of claim 8, wherein the at least one interior layer further comprises at least one focal point selected from triazole, gallic acid, an amino acid, a peptide, a hydrocarbon, a linear polymer of an oligonucleotide.

13. The dendrimer of claim 8, wherein the repeating units are selected from polyethylene glycol, an amino acid, a peptide, a hydrocarbon, a linear polymer of an oligonucleotide.

14. The dendrimer of claim 8, wherein the dendrimer includes 1 to 15 interior layers.

15. A dendrimer comprising one to ten generations, wherein at least one of generation 1 to generation 10 includes 2-deoxyglucose groups or trehalose groups, wherein the —OH of the 2-deoxyglucose groups or the —OH of the trehalose groups are not attached to any other moiety or atom, or a fraction of the —OH are attached to another molecule, or all of the —OH groups are attached to another molecule.

16. A dendrimer comprising:
generation 1 comprising a polyamidoamine bearing hexyne arms;
generation 2 comprising a gallic acid building blocks-based layer; and
at least one additional generation comprising 2-deoxyglucose surface groups or trehalose surface groups.

17. The dendrimer of claim 16, wherein generation 3, generation 4, or generation 5 comprises the 2-deoxyglucose surface groups or trehalose surface groups.

18. A composition comprising a 2-deoxyglucose-terminated dendrimer configured around a 2-armed core to 200-armed core.

19. A composition comprising a trehalose-terminated dendrimer configured around a 2-armed core to 200-armed core.
